(19) United States
(12) Patent Application Publication
(10) Pub. No.: US 2006/0281670 A1
(43) Pub. Date: Dec. 14, 2006

(54) Compositions and methods for modulating angiogenesis

(75) Inventors: Emery H. Bresnick, Madison, WI (US); Soumen Paul, Madison, WI (US)

Correspondence Address: GODFREY & KAHN, S.C., 780 N. WATER STREET, MILWAUKEE, WI, 53202, US

(73) Assignee: Wisconsin Alumni Research Foundation (WARF)

(21) Appl. No.: 11/450,502

(22) Filed: Jun. 9, 2006

Related U.S. Application Data
(60) Provisional application No. 60/689,413, filed on Jun. 10, 2005.
(60) Provisional application No. 60/724,104, filed on Oct. 6, 2005.

(51) Int. Cl. A61K 38/17; A61K 38/22; A61K 38/12

(52) U.S. Cl. 514/9; 514/12; 514/15

(57) ABSTRACT

Methods and compositions for modulating angiogenesis are disclosed. Such modulation is made possible by the use of NK-B, NK-B analogs, NK receptor agonists and NK receptor antagonists to promote or inhibit angiogenesis. The method for modulating the angiogenic activity of cells comprises contacting cells capable of angiogenesis with an effective amount of NK-B, an NK-B analog, an NK receptor agonist or an NK receptor antagonist wherein the angiogenic activity of the cells is increased or decreased. The angiogenesis modulating compounds can be administered to alleviate and or prevent angiogenesis related diseases in patients, such as, for example, cancer, rheumatoid arthritis, macular degeneration, atherosclerosis, coronary artery disease, peripheral vascular disease, varicose veins and preeclampsia.

### REFERENCE TO RELATED PATENT APPLICATION

This non-provisional application claims the benefit of U.S. Provisional application 60/689,413, filed Jun. 10, 2005, and 60/724,104 filed Oct. 6, 2005, each of which is incorporated herein by reference in its entirety.

### STATEMENT AS TO RIGHTS TO INVENTIONS MADE UNDER FEDERALLY SPONSORED RESEARCH AND DEVELOPMENT

This work was supported in part by a grant from the National Institutes of Health—DK50107/DK55700. The Government of the United States of America may have certain rights in this invention.

### FIELD OF THE INVENTION

This invention relates generally to compositions and methods for modulating angiogenesis. In particular, the present invention is directed to the use of neurokinin B (NK-B), and neurokinin receptor agonists and antagonists for promoting or inhibiting blood vessel morphogenesis.

### BACKGROUND OF THE INVENTION

The development of new blood vessels from existing vasculature termed angiogenesis is an essential physiological process and is a critical pathological development in certain disease states including rheumatoid arthritis, diabetic retinopathy, macular degeneration, atherosclerosis, psoriasis, and tumor growth/metastasis (Carmeliet and Jain, Nature. (2000) 14;407(6801):249-57; Folkman, Annu Rev Med. (2006);57:1-18; Hanahan and Weinberg, Cell (2000) 100(1):57-70 2000). Conversely, inhibition of blood vessel development characterizes other disease states such as, coronary artery disease, peripheral vascular disease and the pregnancy-associated disorder preeclampsia. Multiple endogenous factors have been implicated in promoting and suppressing angiogenesis, and a balance between pro- and anti-angiogenic activities determines the angiogenic response.

Endogenous angiogenesis promoters include vascular endothelial growth factor (VEGF) and basic fibroblast growth factor (bFGF). These compounds induce capillary growth and, in the case of tumors supply nutrients allowing the tumor to grow. In the case of diseases of the eye, such as retinopathy and macular degeneration, blood vessel proliferation is stimulated where there should be an absence of vascularization.

Endogenous angiogenesis inhibitors target endothelium via suppressing cell proliferation and migration, inducing apoptosis, downregulating pro-angiogenic factors and signaling pathways, and inducing anti-angiogenic factors (Folkman, 2006). Such endogenous inhibitors include angiostatin, endostatin and tumstatin. Endogenous angiogenesis inhibitors can antagonize cell surface integrins (Sund et al., (2005) Proc. Natl. Acad. Sci. U.S.A. 102:2934-2939). Novel anti-angiogenic mechanisms include impaired tubulin polymerization (Mabjeesh et al., (2003) Cancer Cell. 3:363-75), inhibition of ATP synthase (Moser et al., (2001) Proc Natl Acad Sci U S A. 98:6656-61), and induction of VEGF receptor proteolysis (Cai et al., (2006) J Biol Chem. 281:3604-13).

Both intact proteins and proteolytic fragments can be endogenous angiogenesis inhibitors. Whereas plasminogen, type XVIII collagen (Col XVIII), Col XV, Col IVα1, Col IVα2, Col IVα3, and fibronectin lack anti-angiogenic activity, proteolytic cleavage of these proteins yields angiostatin, endostatin, endostatin-like fragment from type XV collagen, arresten, canstatin, tumstatin, and anastelin respectively, which are anti-angiogenic (Nyberg et al., (2005) Cancer Res. 65:3967-79). Thrombospondin 1 exemplifies an anti-angiogenic protein that functions as an intact protein (Sund et al., 2005). A protein precursor and proteolytic product can both be anti-angiogenic as illustrated by calreticulin and its N-terminal fragment vasostatin (Pike et al., (1999) Blood. 94:2461-2468).

Under normal physiological conditions, humans or animals undergo angiogenesis only in very specific restricted situations. For example, angiogenesis is normally observed in wound healing, fetal and embryonal development and formation of the corpus luteum, endometrium and placenta. It has been reported that new vessel growth is tightly controlled by many angiogenic regulators (see, e.g., Folkman, J., Nature Med., (1995)1:27-31), and the switch of the angiogenesis phenotype depends on the net balance between up-regulation of angiogenic stimulators and down-regulation of angiogenic suppressors. However, in pathological conditions, such as various cancers, ocular diseases etc. as described above, the switch to an angiogenic phenotype becomes a vicious circle in which the pathologic state induces increased angiogenesis thereby providing increased nutrients to the diseased cells allowing increased proliferation and further increasing angiogenesis.

If this angiogenic activity could be repressed or eliminated, then the tumor, although present, would not grow. There are many reports suggesting that inhibiting tumor angiogenesis should provide a practical approach to long-term control of the disease. Blocking positive regulators of angiogenesis or utilizing negative regulators to suppress angiogenesis results in a delay or regression of experimental tumors. Moreover, in the disease state, prevention of angiogenesis could avert the damage caused by the invasion of the new micro vascular system effectively.

In contrast, the pregnancy-associated disorder preeclampsia is a disease resulting in a decrease in angiogenesis. Preeclampsia affects approximately 5% of pregnant women and leads to significant mortality and morbidity (Redman and Sargent, Science. 2005 Jun. 10;308(5728):1592-4). During normal placental development, trophoblast cells migrate into the spiral arterioles of the placental vasculature. It is proposed that incorporation of trophoblast cells into the maternal vasculature allows for increased perfusion to meet the nutrient demands of the developing fetus. In preeclampsia, trophoblast cell migration is impaired, which correlates with hypoxia. The hypoxic placenta gives rise to factor(s) that oppose vascular remodeling and deregulate vascular function systemically (Roberts and Lain, Placenta. 2002 May;23(5):359-72). Thus, defective remodeling of the placental vasculature and systemic vascular deregulation are hallmarks of preeclampsia.

Consequently, an ability to modulate angiogenesis so as to promote growth of new vasculature, such as, for example, in the case of preeclampsia or to inhibit the growth of new vasculature such as, for example, in the case of cancer and macular diseases would be highly desirable. Further, if the means of regulating relied on the ability to modulate or perturb a coherent regulatory axis, there would be a greater facility to provide a beneficial outcome.

### SUMMARY OF THE INVENTION

The present invention provides methods and compositions for modulating angiogenesis in cells capable of angiogenesis. Such angiogenesis modulation is made possible by the use of NK-B, NK-B analogs, NK receptor agonists and NK receptor antagonists to promote or inhibit angiogenesis. The method for modulating the angiogenic activity of cells comprises contacting cells capable of angiogenesis with an effective amount of NK-B, an NK-B analog, an NK receptor agonist or an NK receptor antagonist wherein the angiogenic activity of the cells is increased or decreased. The angiogenesis modulating compounds can be administered to alleviate and or prevent angiogenesis related diseases, such as, for example, cancer, rheumatoid arthritis, macular degeneration, atherosclerosis, coronary artery disease, peripheral vascular disease, varicose veins and preeclampsia. In some preferred embodiments, the method of modulating angiogenesis comprises administering an NK receptor antagonist resulting in an increase in angiogenesis. In preferred embodiments, the antagonist is selected from the group consisting of: L733060, CP99994, MK869, SDZ NKT 343, GR 82334, L-732,138, RP 67580, Spantide I, WIN51708, SR142801, SB235375, SB218795, SB222200 and combinations thereof.

In other exemplary embodiments of the invention, the method of modulating angiogenesis comprises administering NK-B or an NK-B analog or an NK receptor agonist resulting in a decrease in angiogenesis. In particularly preferred embodiments, the NK receptor agonist is selected from the group consisting of: NK-B (SEQ ID NO: 1), cycloseptide, C14TKL-1, GR 73632, hemokinin and [Sar⁹-Met(O₂)¹¹]-Substance P, senktide, [MePhe7] neurokinin B. In various exemplary embodiments the methods according to the invention can be carried out in vivo or in vitro.

The method according to the invention also includes pharmaceutical compositions. When the invention includes a pharmaceutical composition, the pharmaceutical composition includes (a) an effective amount of NK-B, an NK-B analog, an NK receptor agonist or an NK receptor antagonist and (b) a pharmaceutically acceptable carrier. In some preferred embodiments, when the pharmaceutical composition includes an NK receptor antagonist, angiogenesis is increased in the patient. In other preferred embodiments, when the pharmaceutical composition includes NK-B, an NK-B analog or an NK receptor agonist, the method results in a decrease in angiogenesis.

In some preferred embodiments, when the pharmaceutical composition includes an NK receptor antagonist, the NK receptor antagonist is L733060, CP99994, MK869, SDZ NKT 343, GR 82334, L-732,138, RP 67580, Spantide I, WIN51708, SR142801, SB235375, SB218795, SB222200, mNK-B. In other preferred embodiments, the invention composition contains NK-B (SEQ ID NO: 1) or an NK-B agonist that may include, e.g. cycloseptide, C14TKL-1, GR 73632, hemokinin and [Sar⁹-Met(O₂)¹¹]-Substance P, senktide, [MePhe7] neurokinin B or combinations thereof resulting in a decrease in angiogenesis in the patient.

The composition can be is administered in any effective manner, such as, for example, orally, rectally, subcutaneously, parenterally, transdermally, topically or via a timed release implant.

In various exemplary embodiments directed to therapeutic treatment, the patient in need suffers from a disease in which there is a perturbation in angiogenesis. Such diseases may include, for example, rheumatoid arthritis, diabetic retinopathy, macular degeneration, atherosclerosis, psoriasis, tumor growth/metastasis, coronary artery disease, peripheral vascular disease, varicose veins and preeclampsia.

In yet another exemplary embodiment the invention encompasses a method for inhibiting blood vessel morphogenesis comprising contacting cells capable of blood vessel morphogenesis with a blood vessel morphogenesis-inhibitory amount of an isolated neurokinin-B peptide (SEQ ID NO: 1), analogs thereof, an NK receptor agonist or an NK receptor antagonist. In some embodiments the cells capable of blood vessel morphogenesis are endothelial cells. In various embodiments the method is carried out in vivo or in vitro. In some embodiments, the cells capable of blood vessel morphogenesis promote or maintain a pathologic state upon forming blood vessels. In these embodiments, the pathologic state is cancer, rheumatoid arthritis, hemangioma, psoriasis, or ocular disease.

In another preferred embodiment, the invention is directed to the use of NK-B (SEQ ID NO: 1), an NK-B analog, an NK receptor agonist, or an NK receptor antagonist in the manufacture of a medicament for the treatment of an angiogenesis related condition. In this embodiment, the medicament is used, for example, in treating rheumatoid arthritis, diabetic retinopathy, macular degeneration, atherosclerosis, psoriasis, tumor growth/metastasis, coronary artery disease, peripheral vascular disease, varicose veins or preeclampsia. The NK receptor agonist or antagonist is e.g., cycloseptide, C14TKL-1, GR 73632, hemokinin and [Sar⁹-Met(O₂)¹¹]-Substance P, senktide, [MePhe7] neurokinin B, L733060, CP99994, MK869, SDZ NKT 343, GR 82334, L-732,138, RP 67580, Spantide I, WIN51708, SR142801, SB235375, SB218795, SB222200 or combinations thereof.

These and other features and advantages of various exemplary embodiments of the invention are described in, or are apparent from the following detailed description of various exemplary embodiments of the methods and compositions according to this invention.

### BRIEF DESCRIPTION OF THE FIGURES

Various exemplary embodiments of the methods of this invention will be described in detail, with reference to the following figures, wherein:

FIGS. 1A-G are data showing that NK-B reversibly opposes endothelial cell vascular network assembly. FIG. 1A, is a graph of the results of YSEC proliferation assays mean+/−S.E., 3 independent experiments. FIG. 1B, is a graph showing the results of YSEC motility assay (mean+/−S.E., 4 independent experiments). FIG. 1C, are representative micrographs of YSEC vascular network assembly assays. FIG. 1D, are representative micrographs showing reversibility of vascular network assembly blockade. FIG. 1E, are representative micrographs showing cell selectivity of NK-B inhibition of vascular network assembly as shown in HAECs, HUVECs and HMVECs treated with IBMX or NK-B/IBMX and incubated for 12 h. FIG. 1F, are histograms showing the results of quantitative real-time RT-PCR analysis of NK1, NK2, and NK3 receptor mRNA levels in HUVECs, HAECs, and HMVECs. The relative mRNA levels were normalized by HPRT mRNA levels (mean+/−S.E., three independent experiments). FIG. 1G is a histogram showing the effects of IBMX, forskolin and NK-B on cAMP concentration in HUVECs, HAECs, HMVECs. FIG. 1H is a plot of the change in cAMP in YSECs treated with NK-B or NK-B/IBMX (mean+/−S.E., 3 independent experiments).

FIGS. 2A and B are data showing that NK-B is anti-angiogenic in vivo and disruption of endogenous neurokinin signaling is pro-angiogenic in the chick chorioallantoic membrane model. FIG. 2A are representative micrographs at 40× magnification, of vascularization in chick embryos 48 h after treatment as indicated. Top left panel, methylcellulose containing vehicle; top right panel, FGF2 (500 ng)/IBMX (100 μg); middle left panel, FGF2/NK-B (40 μg); middle right panel FGF2/NK-B (40 μg) with IBMX; lower left panel, FGF2/mNK-B/IBMX (mNK-B, inactive mutant of NK-B); lower right panel, a combination of NK1-(L733060, 5 μM) and NK3-(SB222200, 2 μM) selective inhibitors; each treatment applied to the CAM of day 7 chicken embryos. FIG. 2B is a histogram showing the relative microvasculature density in each treatment of FIG. 2A as determined by overlaying a grid on the Adobe Illustrator images.

FIGS. 3A-C are data showing the NK receptor requirement for NK-B-mediated abrogation of vascular network assembly. FIG. 3A, is a histogram showing the relative mRNA levels quantitative RT-PCR analysis of NK1 (left panel) and NK3 (right panel) mRNA in YSEC clonal lines stably expressing NK1 and NK3 siRNA molecules. (RT, reverse transcriptase). FIG. 3B, are representative micrographs of cells treated in FIG. 3A showing intermediate abrogation of vascular network assembly assay in either NK1 or NK3 knockdown YSEC cells. Control, left panels; NK1 knockdown, middle panels; and NK3 knockdown, right panels. FIG. 3C, YSECs were treated with IBMX (left panel); NK-B/IBMX (middle panel); and NK1-(L733060) and NK3-(SB222200) selective inhibitors for 15 min. Inhibition of both NK1 and NK3 abolishes NK-B inhibition on vascular assembly.

FIGS. 4A-D are data elucidating the NK-B signaling circuitry. FIG. 4A, real-time measurements of Ca⁺² oscillations in YSEC cells treated as indicated. Ca⁺² oscillation patterns are plotted as a percentage of fluorescence intensity at time 0 (F₀), of two representative cells (2 independent experiments per condition) (Sup, supplemented M200 medium). FIG. 4B is a histogram of quantitative analysis of Ca⁺² oscillation patterns as a percentage of cells exhibiting Ca⁺² oscillations (mean+/−SE, 3 independent experiments). FIG. 4C shows Western blots of YSEC lysates analyzed for phosphorylated and nonphosphorylated forms of FAK kinase, p42/44 MAPK, and Akt. Cells were treated with IBMX or NK-B/IBMX in supplement-free medium for 1 h, and plated on Matrigel containing supplemented medium for 0.5, 1, 2, and 5 h. Cells continuously grown in the presence of supplemented medium were used as a control. FIG. 4D, is a schematic illustrating proposed NK-B signaling crosstalk.

FIGS. 5A-E, data showing NK-B downregulates type I and type II VEGF receptors. FIG. 5A are histograms of real-time RT-PCR quantification of relative mRNA levels of YSECs and HUVECs treated with IBMX or NK-B/IBMX and plated on Matrigel containing supplemented medium for 0.5, 1, 2, and 5 h. The transcript level in untreated cells was designated 1 (2-3 independent experiments). RT, reverse transcriptase. FIG. 5B is a Western blot analysis of Flt-1 and Flk-1 in YSECs and HUVECs. FIG. 5C are histograms of relative mRNA levels of Flt-1 (left panel), Flk-1 (middle panel) in YSECs treated with IBMX, NK-B/IBM X, NK-B/IBMX/VEGF and NK-B/IBMX/FGF2 showing that VEGF, but not FGF2, rescues Flt-1 and Flk-1 expression and cell signaling. The right panel shows the relative mRNA levels of FGFR1 in YSEC cells treated with IBMX, IBMX/FGF-2 and NK-B/IBMX/FGF2 data show that FGFR1 transcription is increased in presence of FGF2 with or without NK-B. FIG. 5D is a Western blot analysis of unphosphorylated and phosphorylated FAK and p42/p44 MAPK in cells analyzed in FIG. 5C. FIG. 5E, are representative micrographs of YSECs treated with IBMX, NK-B/IBMX, NK-B/IBMX/recombinant VEGF164 (100 ng/ml) and NK-B/IBMX and mouse FGF2 (100 ng/ml).

FIG. 6A-D, are data showing that NK-B increases synthesis of the anti-angiogenic protein calreticulin. FIG. 6A are SDS-PAGE gels of YSEC whole cell extracts that were treated with IBMX or NK-B/IBMX for 1 h in supplement-free M200 medium and then 10 min in supplemented M200 medium and subjected to 2D gel electrophoresis and stained with Coomassie blue; the inset shows the spot identified as calreticulin. FIG. 6B, Western blot analysis of calreticulin in whole cell lysates of YSECs after treating as in FIG. 6A. Blots were probed for calreticulin, and then stripped and reprobed for α-tubulin. FIG. 6C, SDS-PAGE of E. coli overexpressed and purified recombinant calreticulin and vasostatin. FIG. 6D, are representative micrographs of YSECs and HUVECs treated, as shown, with GST (10 μg/ml), calreticulin (13 μg/ml), or vasostatin (5 μg/ml). Calreticulin and vasostatin inhibit vascular assembly in both cell types.

FIG. 7A-C, NK-B and TXA-2 signaling synergistically opposes vascular network assembly. FIG. 7A are representative micrographs of YSECs, HUVECs, and HAECs treated with vehicle, NK-B, U46619 (20 μM), or NK-B/U46619 in supplement-free medium for 1 or 2 h, respectively, plated on Matrigel containing supplemented medium, and incubated for 16 h at 37° C. to assess vascular network assembly. FIG. 7B, is a graph of cAMP concentration of YSECs treated with NK-B, U46619, or NK-B/U46619 in supplement-free M200 medium, and cAMP was quantitated various times thereafter (mean+/−S.E., 3 independent experiments). FIG. 7C, is a graph of real-time measurements of Ca⁺² oscillations in YSECs. The left panel depicts the % of cells exhibiting oscillations. The right panel shows fluorescence intensity at different time intervals (F) relative to fluorescence intensity at time 0 (F₀) (Sup, supplemented M200 medium).

FIG. 8 is a schematic illustrating the anti-angiogenic NK-B/TXA2 regulatory axis.

FIG. 9 are representative micrographs showing that NK-B alone decreases the kinetics of vascular network assembly. Digital images were captured at 6 and 20 h, and representative images are shown.

FIG. 10 are micrographs showing that NK-B/IBMX abrogates vascular network assembly in three-dimensional collagen gels.

FIG. 11 are representative digital images of chick embryos showing, in vivo, that VEGF164-dependent angiogenesis (top panel) dominates over the anti-angiogenic activity of NK-B/IBMX (bottom panel).

FIG. 12 are representative micrographs showing that forskolin only partially disrupts YSEC vascular network assembly and does not affect HUVEC vascular network assembly.

FIGS. 13A and 13B are data illustrating that forskolin, but not calreticulin and vasostatin, downregulates Flt-1 and Flk-1. FIG. 13A, is a graph of relative mRNA levels of Flt-1 (left panel) and Flk-1 (right panel) of YSECs treated with vehicle, IBMX (100 μM), forskolin (10 μM)/IBMX, calreticulin (13 μg/ml) and vasostatin (5 μg/ml) in supplement-free M200 medium for 1 h. RNA from untreated cells was used as a control, and the expression level in untreated cells was designated as 1 (mean, 2 independent experiments). RT, reverse transcriptase. FIG. 13B, is a Western blot of calreticulin in YSEC whole cell extracts. Data indicate that Ca⁺² ionophore ionomycin weakly increases calreticulin synthesis. The calreticulin/alpha-tubulin ratio increased ˜2-fold upon ionomycin treatment.

FIGS. 14A-C are micrographs of chick embryos showing NK-inhibitor induced angiogenesis in the chorioallantoic membrane in vivo. FIGS. 14A-C demonstrate the ability of the NK3 receptor antagonist to induce angiogenesis on chick chorioallantoic membrane.

FIG. 15 are micrographs illustrating the results of three experiments with various treatments showing that NK-B prevents endothelial cell tube formation.

FIGS. 16A and B are micrographs illustrating the effects of NK-B on endothelial tube formation. FIG. 16A, endothelial tube formation occurs in absence of NK-B (top panel); IBMX+NK-B results in inhibition of endothelial tube formation (middle panel); NK-B endothelial tube inhibition is reversible after NK-B; endothelial tube spontaneously reforms following NK-B washout. FIG. 16B, shows that NK-B does not block the maintenance signal for already formed tubes.

FIGS. 17A-C are data showing that the effect of NK-B is specific to endothelial cells. FIG. 17A, HAEC (top), HUVEC (middle) and HMVEC (bottom) were treated with IBMX alone or NKB+IBMX. Endothelial tube formation was inhibited by NK-B but not IBMX in HAEC and HUVEC but not HMVEC. FIG. 17B, RT-PCR analysis of neurokinin receptor subtypes NK1, NK2, and NK3 levels in HUVEC cells, HAEC cells and HMVEC cells. FIG. 17C are data showing cAMP levels in HUVEC, HAEC and HMVEC cells treated with IBMX, forskolin and NK-B.

FIG. 18A-C are data illustrating that NK-B mediates function through NK receptors. FIG. 18A, YSEC cells plated on Matrigel in the presence of: solvent and IBMX only (top); NK-B and IBMX (middle) and NK1 and NK3 inhibitors plus NK-B and IBMX. FIG. 18B, quantitative RT-PCR analysis of NK1 (left) and NK3 (right) mRNA in stable clones of YSEC cells expressing NK1 and NK3 SiRNA molecules (RT=reverse transcriptase). FIG. 18C, endothelial tube formation with stable clones of YSEC cells analyzed in FIG. 18B showing that knockdown of receptor partially remediates effect of NK-B on endothelial tube formation.

FIGS. 19A and B are data illustrating that NK-B prevents endothelial cell migration. FIG. 19A are photographs of YSEC cells at 0 min., 60 min. and 120 min. treated with solvent, IBMX (μM), IBMX+NK-B (100 μM), IBMX+NK-B+NK1 inhibitor (2.5 μM)+NK3 inhibitor (1 μM) and IBMX+forskolin for 1 h and plated on Matrigel. FIG. 19B, histogram quantifying data shown in FIG. 19A.

FIG. 20 is data showing that NK-B mediates induction of calreticulin in endothelial cells. FIG. 20A, YSEC cells were treated either with IBMX (left panel) or IBMX+NK-B (right panel). Middle inset shows calreticulin band. FIG. 20B, Western blot analysis in whole cell extracts treated as shown.

FIGS. 21A-C are data showing NK-B inhibits VEGF receptor transcription and downstream signaling. FIG. 21A, YSEC cells were treated with IBMX or IBMX+NK-B in unsupplemented media for 1 h. Treated cells were then plated on Matrigel in presence of supplemented media for 0.5 h, 1 h, 2 h, and 5 h. Plots show the effect on transcription of VEGF, Flt-1, Flk-1, E. Cadherin, Notch-1 and Notch-4. FIG. 21B, are histograms showing effects of similar experiments to FIG. 21A but with HUVEC cells; data normalized by HPRT level. FIG. 21C, are Western blots of the same cells shown in FIG. 21A.

FIG. 22 are micrographs showing that VEGF but not bFGF rescues NK-B effect on YESC cells. Panels on left are taken at 20 h, 40 h on right. Cells treated with IBMX, IBMX+VEGF, IBMX+NK-B, IBMX+NK-B+VEGF and IBMX+NK-B+bFGF.

FIG. 23A-E illustrates that NK-B suppresses endothelial cell motility and vascular network assembly. FIG. 23A, histogram showing cell proliferation of YSECs and HUVECs plated in supplement-free medium for 24 h and treated with vehicle, or NK-B for 1 h. FIG. 23B, histogram showing cell migration of YSECs and HUVECs cells plated in supplement-free medium for 24 h and treated with vehicle, or NK-B for 1 h. FIG. 23C is a histogram showing the rate of migration of cells treated as in FIGS. 23A and B. FIG. 23D, representative micrographs of YSECs and HUVECs treated with vehicle or NK-B for 1 and 2 h respectively; FIG. 23E, are histograms illustrating length of tubular structures from three adjacent frames quantified from 3 independent structures.

FIGS. 24A-G, IBMX and TXA2 potentiate NK-B activity to regulate endothelial cell function. FIG. 24A, is a graph showing the cAMP concentration as a function of time in YSEC cells treated with NK-B, IBMX or NK-B/IBMX in supplement free media. FIG. 24B is a histogram showing percent increase in cell proliferation of YSECs treated with NK-B or NK-B/IBMX in supplement-free medium (MEAN+/−S.E. of 3 independent experiments, p<0.05). FIG. 24C, is a histogram showing migration of cells treated as in FIG. 24B expressed as percent of total cells (mean+/−S.E., 3 independent experiments. FIG. 24D, are micrographs showing YSECs treated with vehicle, IBMX, or NK-B/IBMX for 1 h in supplement-free medium, plated on Matrigel containing supplemented medium, and incubated for 20 h at 37° C. FIG. 24E, is a graph of cAMP concentrations of YSEC cells treated with NK-B, U46619, or NK-B/U46619. FIG. 24F, are micrographs showing YSEC cells treated with U46619 or NK-B/UB6619 and plated on Matrigel. FIG. 24G is a histogram quatifying the data obtained in FIG. 24F.

FIGS. 25 A-C display data showing that NK-B inhibits vascular remodeling of certain human endothelial cell subtypes. FIG. 25A, are micrographs showing HUVEC, HAEC and HMVEC cells treated with IBMX or NK-B/IBMX for 2 h in supplement-free medium, plated on Matrigel and incubated for 12 h. FIG. 25B, are histograms from quantitative real-time RT-PCR analysis of NK1 (left), NK2 (middle) and NK3 (right) receptor mRNA levels in HUVEC, HAEC and HMVEC cells. Values are normalized by HPRT mRNA levels (mean+/−S.E., 3 independent experiments). FIG. 25C is a histogram showing cAMP concentration in HUVEC, HAEC and HMVEC cells treated with IBMX with or without forskolin or NK-B for 1 h. cAMP was quantitated in cell lysates (mean+/−S.E., 3 independent experiments).

FIG. 26A-C display data showing an NK receptor requirement for NK-B-mediated abrogation of vascular network assembly. FIG. 26A are histograms showing relative mRNA levels of NK1 and NK3 in YSEC clonal lines stably expressing NK1 (left panel) and NK3 (right panel) siRNA molecules (RT, reverse transcriptase). FIG. 26B, are micrographs showing control (vehicle) and siRNA expressing YSEC cells treated with IBMX or NK-B/IBMX incubated on Matrigel for 20 hh at 37° C. Knocking-down either NK1 or NK3 reduced the capacity of NK-B/IBMX to abrogate vascular network assembly. FIG. 26C, are micrographs showing YSEC cells treated with IBMX or NK-B/IBMX with or without NK1-(1733060) and NK3-(SB222200) selective inhibitors for 15 minutes followed by NK-B/IBMX, and plated on Matrigel to assess vascular network assembly.

FIG. 27A-C are data showing that NK-B downregulates VEGF receptors. FIG. 27A, are histograms showing relative mRNA levels in YSEC cells (top row) and HUVEC cells (bottom row) and treated with Fltl (left column); Flk-1 (middle column) and FGFR1 (right column). FIG. 27B, are Western blot analysis of YSEC CELLS and HUVEC cells treated as shown. FIG. 27C, are histograms showing transcript level, quantified by real-time RT-PCR, of YSEC cells (top row) and HUVEC cells (bottom row) treated with IBMX or NK-B/IBMX and plated on Matrigel for 0.5, 1, 2, and 5 hours (RT, reverse transcriptase).

### DETAILED DESCRIPTION OF THE INVENTION

I. In General

Before the present materials methods are described, it is understood that this invention is not limited to the particular methodology, protocols, cell lines, and reagents described, as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by” and “having” can be used interchangeably.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The practice of the present invention will employ, unless otherwise indicated, conventional techniques of molecular biology, microbiology, recombinant DNA, and immunology, which are within the skill of the art. Such techniques are explained fully in the literature. See, for example, Molecular Cloning A Laboratory Manual, 2nd Ed., ed. by Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); and Handbook Of Experimental Immunology, Volumes I-IV (D. M. Weir and C. C. Blackwell, eds., 1986).

As used herein, “subject” or “patient” means mammals and non-mammals. “Mammals” means any member of the class Mammalia including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, and the like. The term “subject” does not denote a particular age or sex.

As used herein, “administering” or “administration” includes any means for introducing the respective substance into the body, preferably into the systemic circulation. Administration routes include but are not limited to, rectal, oral; buccal, sublingual, pulmonary, transdermal, transmucosal, as well as subcutaneous, intraperitoneal, intravenous, and intramuscular injection.

As used herein, “pharmaceutical composition” means therapeutically effective amounts of the angiogenic agent together with suitable diluents, preservatives, solubilizers, emulsifiers, and adjuvants, collectively “pharmaceutically-acceptable carriers.” As used herein “pharmaceutically acceptable carriers” are well known to those skilled in the art and include, but are not limited to, 0.01-0.1M and preferably 0.05M phosphate buffer or 0.9% saline. Additionally, such pharmaceutically acceptable carriers may be aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

The phrase “effective amount,” as used herein, means an amount of an agent which is sufficient enough to significantly and positively modify symptoms and/or conditions to be treated (e.g., provide a positive clinical response). The effective amount of an active ingredient for use in a pharmaceutical composition will vary with the particular condition being treated, the severity of the condition, the duration of the treatment, the nature of concurrent therapy, the particular active ingredient(s) being employed, the particular pharmaceutically-acceptable excipient(s)/carrier(s) utilized, and like factors within the knowledge and expertise of the attending physician. In general, the use of the minimum dosage that is sufficient to provide effective therapy is preferred. Patients may generally be monitored for therapeutic effectiveness using assays suitable for the condition being treated or prevented, which will be familiar to those of ordinary skill in the art.

II. The Invention

The inventor's studies to dissect genetic networks instigated by the transcription factor GATA-1 in erythroid cells revealed that GATA-1 activates transcription of the murine preprotachykinin-B gene (Tac-2) (Pal et al., 2004 J. Biol. Chem. 279:31348-31356.), which encodes neurokinin-B (NK-B) precursor protein. The human ortholog TAC-3 is highly induced upon ex vivo differentiation of peripheral blood hematopoietic precursors (Pal et al., 2004). Tac-2 is also expressed by neurons, the uterus, and syncytiotrophoblasts of the placenta. Biological functions of tachykinins include smooth muscle contraction, vasodilation, neurotransmission, neurogenic inflammation, and immune system activation. NK-B activates NK1, NK2, and NK3 G-protein coupled receptors (Fong et al. J. Biol. Chem. 267:25664-25667., 1992). As quantitative RT-PCR analysis did not reveal NK receptor expression by erythroid cells, erythroid cell-derived NK-B might act on neighboring cells within the hematopoietic and/or vascular microenvironments. NK receptors are expressed on mouse yolk sac and aortic endothelial cells, and NK-B induces cAMP accumulation in these cells (Pal et al., 2004). NK receptors are also expressed on endothelial cells of rat post capillary venules (Bowden et al., 1994 Proc. Natl. Acad. Sci. U.S.A. 91:8964-8968), human umbilical vein endothelial cells (HUVECs) (Brownbill et al., 2003 J. Clin. Endocinol. Metab. 88:2164-2170), and bovine corpus luteal endothelial cells (Brylla et al., 2005 Regul. Pept. 125:125-133). These findings led the inventors to the realization that NK-B regulatory axis may have specific affects on endothelial cells.

The present invention provides methods and compositions for modulating angiogenesis in cells capable of angiogenesis. Such angiogenesis modulation is made possible by the use of NK-B, NK-B analogs, NK receptor agonists and NK receptor antagonists to promote or inhibit angiogenesis. The method for modulating the angiogenic activity of cells comprises contacting cells capable of angiogenesis with an effective amount of NK-B, an NK-B analog, an NK receptor agonist or an NK receptor antagonist wherein the angiogenic activity of the cells is increased or decreased. The angiogenesis modulating compounds can be administered to alleviate and or prevent angiogenesis related diseases, such as, for example, cancer, rheumatoid arthritis, macular degeneration, atherosclerosis, coronary artery disease, peripheral vascular disease, varicose veins and preeclampsia.

As used herein, the term “NK-B modulating agent” or “NK-B modulating compound” means a molecule that perturbates or modulates the NK-B regulatory axis. Such molecules may be any molecule that results in NK-B mediated anti-angiogenic or pro-angiogenic effects. Such molecules include, but are not limited to, NK-B itself, NK-B analogs, NK-B potentiator substances, NK-B agonists or NK-B antagonists.

According to one exemplary embodiment, the invention comprises a method for modulating the angiogenic activity of cells comprising contacting cells capable of angiogenesis with an effective amount of NK-B, an NK-B analog, an NK receptor agonist or an NK receptor antagonist wherein the angiogenic activity of the cells is increased or decreased. In some preferred embodiments, the method of modulating angiogenesis comprises administering an NK receptor antagonist resulting in an increase in angiogenesis. In preferred embodiments, the antagonist is selected from the group consisting of: L733060, CP99994, MK869, SDZ NKT 343, GR 82334, L-732,138, RP 67580, Spantide I, WIN51708, SR142801, SB235375, SB218795, SB222200 and combinations thereof.

In other exemplary embodiments of the invention, the method of modulating angiogenesis comprises administering NK-B an NK-B analogs or an NK-B agonist resulting in a decrease in angiogenesis. In some preferred embodiments, the NK-B agonist is NK-B, an NK-B analog or an NK receptor agonist. In particularly preferred embodiments, the NK-B agonist is selected from the group consisting of: NK-B (SEQ ID NO: 1), cycloseptide, C14TKL-1, GR 73632, hemokinin and [Sar⁹-Met(O₂)¹¹]-Substance P, senktide, [MePhe7] neurokinin B. In various exemplary embodiments the methods according to the invention can be carried out in vivo or in vitro.

In yet another exemplary embodiment the invention encompasses a method for use in inhibiting blood vessel morphogenesis comprising contacting cells capable of blood vessel morphogenesis with a blood vessel morphogenesis-inhibitory amount of an isolated neurokinin-B peptide (SEQ ID NO: 1), analogs thereof, an NK receptor agonist or an NK receptor antagonist. In some embodiments the cells capable of blood vessel morphogenesis are endothelial cells. In various embodiments the method is carried out in vivo or in vitro. In some embodiments, the cells capable of blood vessel morphogenesis promote or maintain a pathologic state upon forming blood vessels. In these embodiments, the pathologic state is cancer, rheumatoid arthritis, hemangioma, psoriasis, or ocular disease.

The method according to the invention also includes pharmaceutical compositions. When the invention includes a pharmaceutical composition, the pharmaceutical composition includes (a) an effective amount of NK-B, an NK-B analog, an NK receptor agonist or an NK receptor antagonist and (b) a pharmaceutically acceptable carrier. In some preferred embodiments, when the pharmaceutical composition includes an NK receptor antagonist, angiogenesis is increased in the patient. In other preferred embodiments, when the pharmaceutical composition includes an NK receptor agonist, the method results in a decrease in angiogenesis.

In some preferred embodiments, when the pharmaceutical composition includes an NK receptor antagonist, the NK receptor antagonist may be e.g., L733060, CP99994, MK869, SDZNKT 343, GR 82334, L-732,138, RP 67580, Spantide I, WIN51708, SR142801, SB235375, SB218795, SB222200 or mNK-B. In other preferred embodiments, the pharmaceutical composition includes an NK receptor agonist, such as, NK-B (SEQ ID NO: 1) or cycloseptide, C14TKL-1, GR 73632, hemokinin and [Sar⁹-Met(O₂)¹¹]-Substance P, senktide, [MePhe7] neurokinin B or combinations thereof resulting in a decrease in angiogenesis in the patient.

The compositions and methods of the invention are directed to therapeutic treatment, as well as, scientific research. As a therapeutic agent, the use of the invention is directed to patients in need thereof. As a research tool, the compositions and methods of the invention are directed to cells, preferably endothelial cells, capable of blood vessel morphogenesis. Cells capable of blood vessel morphogenesis may promote or maintain a pathologic state upon forming blood vessels, including, but not limited to, cancer, rheumatoid arthritis, hemangioma, psoriasis, or ocular disease. Ocular angiogenic diseases are characterized by neovascularization of the cornea and such neovascularization may be the result of Stevens-Johnson syndrome, ocular pemphigoid, corneal injury due to chemical or toxin exposure, virus infection, phlyctenula keratitis, corneal transplant, or contact lens use. Alternatively, cells capable of blood vessel morphogenesis may promote or maintain non-pathologic vascular re-modeling, such endothelial cells involved in wound healing.

Methods according to the invention are equally applicable to either the in vivo or the in vitro setting. For example, the present invention may be applied to inhibit blood vessel formation in a tumor contained within a living subject. Alternatively, the inventive methods may be aimed at inhibiting the formation of blood vessel tubes in cells grown in tissue culture.

In certain embodiments related to anti-angiogenic therapy, the present invention provides methods for inhibiting angiogenesis in a patient. Such methods comprise the step of administering to a patient in need of angiogenesis inhibition an inhibitory amount of an isolated neurokinin B peptide, analog thereof and/or receptor agonist. In preferred embodiments, the active agent utilized in such methods is an isolated neurokinin B peptide having the amino acid sequence Asp-Met-His-Asp-Phe-Phe-Val-Gly-Leu-Met (SEQ ID NO:1). However, in certain other embodiments of the invention, NK-B peptide analogs are used to modulate angiogenesis. NK-B peptide analogs described herein may, but need not, contain additional amino acid residues from those capable of modulating angiogenesis, most preferably the activity of inhibiting blood vessel morphogenesis. Flanking residues may be present on the N-terminal and/or C-terminal side of a peptide analog sequence and may, although not necessarily, act to facilitate internalization, cyclization, purification or other manipulation of the peptide analog. Peptide analogs may further be associated (covalently or noncovalently) with a targeting agent, drug, solid support and/or detectable marker.

In certain other embodiments, related to the promotion of angiogenesis, the present invention provides methods for promoting angiogenesis in a patient. For example, patients in need thereof may include patients with such diseases as, for example, coronary artery disease, peripheral vascular disease and preeclampsia. Such methods comprise the step of administering to a patient in need thereof an angiogenesis promoting amount of an isolated neurokinin B inhibitor and/or receptor antagonist.

Certain preferred modulating agents comprise a peptide in which at least one terminal amino acid residue is modified (e.g., the N-terminal amino group is modified by, for example, acetylation or alkoxybenzylation and/or an amide or ester is formed at the C-terminus). The addition of at least one such group to a linear or cyclic peptide modulating agent may improve the activity of the agent, enhance cellular uptake and/or impair degradation of the agent.

Peptide analogs may be linear or cyclic peptides. A “linear” peptide is a peptide or salt thereof that does not contain an intramolecular covalent bond between two non-adjacent residues. The term “cyclic peptide,” as used herein, refers to a peptide or salt thereof that comprises an intramolecular covalent bond between two non-adjacent residues, forming a cyclic peptide ring that comprises the NK-B sequence. The intramolecular bond may be a backbone to backbone, side-chain to backbone or side-chain to side-chain bond (i.e., terminal functional groups of a linear peptide and/or side chain functional groups of a terminal or interior residue may be linked to achieve cyclization). Preferred intramolecular bonds include, but are not limited to, disulfide bonds; amide bonds between terminal functional groups, between residue side chains or between one terminal functional group and one residue side chain; thioether bonds and(δ₁, δ₁′)-ditryptophan or a derivative thereof. Preferred cyclic peptide modulating agents generally comprise at least eight residues.

As noted above, peptide analogs may comprise polypeptides or salts thereof, containing only amino acid residues linked by peptide bonds, or may additionally contain non-peptide regions, such as linkers. Peptide regions of a NK-B peptide analogs may comprise residues of L-amino acids, D-amino acids, or any combination thereof. Amino acids may be from natural or non-natural sources; α- and β-amino acids are generally preferred. The 20 L-amino acids commonly found in proteins are identified herein by the conventional three-letter or one-letter abbreviations.

An NK-B peptide analog may also contain rare amino acids (such as 4-hydroxyproline or hydroxylysine), organic acids or amides and/or derivatives of common amino acids, such as amino acids having the C-terminal carboxylate esterified (e.g., benzyl, methyl or ethyl ester) or amidated and/or having modifications of the N-terminal amino group (e.g., acetylation or alkoxycarbonylation), with or without any of a wide variety of side-chain modifications and/or substitutions (e.g., methylation, benzylation, t-butylation, tosylation, alkoxycarbonylation) and the like). Preferred derivatives include amino acids having a C-terminal amide group. Residues other than common amino acids that may be present with a modulating agent include, but are not limited to, 2-mercaptoaniline, 2-mercaptoproline, ornithine, diaminobutyric acid, α-aminoadipic acid, m-aminomethylbenzoic acid and α,β-diaminopropionic acid.

A peptide analog or a peptidomimetic of a naturally-occurring NK-B amino acid sequence retains the ability to modulate angiogenesis, preferably the inhibition of blood vessel formation. In general, a peptide analog may contain conservative substitutions such that the ability to modulate a blood vessel morphogenesis is not substantially diminished. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. The critical determining feature of a peptide analog is the ability to modulate a NK-B mediated response. Such ability may be evaluated using assays substantially similar to those provided in the Example section below.

Peptide analogs may further be identified by performing mutational analysis of NK-B and assaying resulting mutants for angiogenesis modulating activity. For example, alanine scanning mutagenesis may be performed to identify key residues necessary for modulating activities. Specifically, the amino acid sequence of NK-B may be submitted to alanine scanning in order to discern the effect of each residue on the ability of the peptide to modulate angiogenesis. Alanine scanning mutagenesis generates a small and systematic set of mutant peptides whose inhibitory activity can be readily tested using the assay techniques set forth herein. Alanine substitution does not impose new structural effects related to hydrogen bonding, unusual hydrophobicity, or steric bulk, and it is expected to cause minimal perturbation of secondary structure; alanine is compatible with all secondary structures in both buried and solvent-exposed positions (Abroi et al., J. Virology, 70(9):6169, 1996; Cunningham et al., Science, 244: 1081, 1989; Rose et al., Science, 229:834, 1985; Klapper et al., Biochem Biophys Res Communic, 78(3):1018, 1977; Chothia C., J. Mol. Biol, 105(1):1, 1976). Also, in contrast to amino acid deletions, substitution with alanine preserves the original spacing of residues. Thus, alanine scanning is an exemplary technique for isolating the effect of particular amino acids within the context of NK-B. Based upon such an analysis, one of skill in the art could prepare peptide analogs which exhibit angiogenesis modulating activity in similar fashion to the naturally-occurring polypeptide sequences.

NK-B analogs useful in the present invention further include “peptidomimetics”. A peptidomimetic useful in the inventive methods is a compound that is structurally similar to a NK-B derived polypeptide, such that the peptidomimetic retains the ability to modulate angiogenesis, preferably the activity of inhibiting blood vessel formation. In general, peptidomimetics are organic compounds that mimic the three-dimensional shape and activity of a particular polypeptide. It is now accepted that peptidomimetics may be designed based on techniques that evaluate three dimensional shape, such as nuclear magnetic resonance (NMR) and computational techniques. NMR is widely used for structural analysis of molecules. Cross-peak intensities in Nuclear Overhauser Enhancement (NOE) spectra, coupling constants and chemical shifts depend on the conformation of a compound. NOE data provide the inter-proton distance between protons through space. This information may be used to facilitate calculation of the lowest energy conformation for the relevant peptide sequence. Once the lowest energy conformation is known, the three-dimensional shape to be mimicked is known. It should be understood that, within embodiments described herein, a peptidomimetic may be substituted for the amino acid sequence of the polypeptide on which the peptidomimetic is based.

Examples of peptidomimetics encompassed by the present invention include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. In addition to rational designing, as described above, a peptidomimetic can be obtained by, for example, screening libraries of natural and synthetic compounds for compounds capable of modulating blood vessel formation.

Peptide-based analogs as described herein may be synthesized by methods well known in the art, including chemical synthesis and recombinant DNA methods. Chemical synthesis may be performed using solution phase or solid phase peptide synthesis techniques, in which a peptide linkage occurs through the direct condensation of the α-amino group of one amino acid with the α-carboxy group of the other amino acid with the elimination of a water molecule. Peptide bond synthesis by direct condensation, as formulated above, requires suppression of the reactive character of the amino group of the first and of the carboxyl group of the second amino acid. The masking substituents must permit their ready removal, without inducing breakdown of the labile peptide molecule.

In solution phase synthesis, a wide variety of coupling methods and protecting groups may be used (see Gross and Meienhofer, eds., “The Peptides: Analysis, Synthesis, Biology,” Vol. 1-4 (Academic Press, 1979); Bodansky and Bodansky, “The Practice of Peptide Synthesis,” 2d ed. (Springer Verlag, 1994)). In addition, intermediate purification and linear scale up are possible. Those of ordinary skill in the art will appreciate that solution synthesis requires consideration of main chain and side chain protecting groups and activation method. In addition, careful segment selection is necessary to minimize racemization during segment condensation. Solubility considerations are also a factor.

Solid phase peptide synthesis uses an insoluble polymer for support during organic synthesis. The polymer-supported peptide chain permits the use of simple washing and filtration steps instead of laborious purifications at intermediate steps. Solid-phase peptide synthesis may generally be performed according to the method of Merrifield et al., J. Am. Chem. Soc. 85:2149, 1963, which involves assembling a linear peptide chain on a resin support using protected amino acids. Solid phase peptide synthesis typically utilizes either the Boc or Fmoc strategy. The Boc strategy uses a 1% cross-linked polystyrene resin. The standard protecting group for α-amino functions is the tert-butyloxycarbonyl (Boc) group. This group can be removed with dilute solutions of strong acids such as 25% trifluoroacetic acid (TFA). The next Boc-amino acid is typically coupled to the amino acyl resin using dicyclohexylcarbodiimide (DCC). Following completion of the assembly, the peptide-resin is treated with anhydrous HF to cleave the benzyl ester link and liberate the free peptide. Side-chain functional groups are usually blocked during synthesis by benzyl-derived blocking groups, which are also cleaved by HF. The free peptide is then extracted from the resin with a suitable solvent, purified and characterized. Newly synthesized peptides can be purified, for example, by gel filtration, HPLC, partition chromatography and/or ion-exchange chromatography, and may be characterized by, for example, mass spectrometry or amino acid sequence analysis. In the Boc strategy, C-terminal amidated peptides can be obtained using benzhydrylamine or methylbenzhydrylamine resins, which yield peptide amides directly upon cleavage with HF.

In the procedures discussed above, the selectivity of the side-chain blocking groups and of the peptide-resin link depends upon the differences in the rate of acidolytic cleavage. Orthogonal systems have been introduced in which the side-chain blocking groups and the peptide-resin link are completely stable to the reagent used to remove the α-protecting group at each step of the synthesis. The most common of these methods involves the 9-fluorenylmethyloxycarbonyl (Fmoc) approach. Within this method, the side-chain protecting groups and the peptide-resin link are completely stable to the secondary amines used for cleaving the N-α-Fmoc group. The side-chain protection and the peptide-resin link are cleaved by mild acidolysis. The repeated contact with base makes the Merrifield resin unsuitable for Fmoc chemistry, and β-alkoxybenzyl esters linked to the resin are generally used. Deprotection and cleavage are generally accomplished using TFA.

Those of ordinary skill in the art will recognize that, in solid phase synthesis, deprotection and coupling reactions must go to completion and the side-chain blocking groups must be stable throughout the entire synthesis. In addition, solid phase synthesis is generally most suitable when peptides are to be made on a small scale.

Acetylation of the N-terminus can be accomplished by reacting the final peptide with acetic anhydride before cleavage from the resin. C-amidation may be accomplished using an appropriate resin such as methylbenzhydrylamine resin using the Boc technology.

Following synthesis of a linear peptide, cyclization may be achieved if desired by any of a variety of techniques well known in the art. Within one embodiment, a bond may be generated between reactive amino acid side chains. For example, a disulfide bridge may be formed from a linear peptide comprising two thiol-containing residues by oxidizing the peptide using any of a variety of methods. Within one such method, air oxidation of thiols can generate disulfide linkages over a period of several days using either basic or neutral aqueous media. The peptide is used in high dilution to minimize aggregation and intermolecular side reactions. This method suffers from the disadvantage of being slow but has the advantage of only producing H₂O as a side product. Alternatively, strong oxidizing agents such as I₂ and K₃ Fe(CN)₆ can be used to form disulfide linkages. Those of ordinary skill in the art will recognize that care must be taken not to oxidize the sensitive side chains of Met, Tyr, Trp or His. Cyclic peptides produced by this method require purification using standard techniques, but this oxidation is applicable at acid pHs. Oxidizing agents also allow concurrent deprotection/oxidation of suitable S-protected linear precursors to avoid premature, nonspecific oxidation of free cysteine.

DMSO, unlike I₂ and K₃ Fe(CN)₆, is a mild oxidizing agent which does not cause oxidative side reactions of the nucleophilic amino acids mentioned above. DMSO is miscible with H₂O at all concentrations, and oxidations can be performed at acidic to neutral pHs with harmless byproducts. Methyltrichlorosilane-diphenylsulfoxide may alternatively be used as an oxidizing agent, for concurrent deprotection/oxidation of S-Acm, S-Tacm or S-t-Bu of cysteine without affecting other nucleophilic amino acids. There are no polymeric products resulting from intermolecular disulfide bond formation. Suitable thiol-containing residues for use in such oxidation methods include, but are not limited to, cysteine, β,β-dimethyl cysteine (penicillamine or Pen), β,β-tetramethylene cysteine (Tmc), β,β-pentamethylene cysteine (Pmc), β-mercaptopropionic aid (Mpr), β,β-pentamethylene-.beta.-mercaptopropionic acid (Pmp), 2-mercaptobenzene, 2-mercaptoaniline and 2-mercaptoproline. Within another embodiment, cyclization may be achieved by amide bond formation. For example, a peptide bond may be formed between terminal functional groups (i.e., the amino and carboxy termini of a linear peptide prior to cyclization), with or without an N-terminal acetyl group and/or a C-terminal amide. Within another such embodiment, the linear peptide comprises a D-amino acid. Alternatively, cyclization may be accomplished by linking one terminus and a residue side chain or using two side chains, with or without an N-terminal acetyl group and/or a C-terminal amide. Residues capable of forming a lactam bond include lysine, ornithine (Orn), α-amino adipic acid, m-aminomethylbenzoic acid, α,β-diaminopropionic acid, glutamate or aspartate.

Methods for forming amide bonds are well known in the art and are based on well established principles of chemical reactivity. Within one such method, carbodiimide-mediated lactam formation can be accomplished by reaction of the carboxylic acid with DCC, DIC, EDAC or DCCI, resulting in the formation of an O-acylurea that can be reacted immediately with the free amino group to complete the cyclization. The formation of the inactive N-acylurea, resulting from O to N migration, can be circumvented by converting the O-acylurea to an active ester by reaction with an N-hydroxy compound such as 1-hydroxybenzotriazole, 1-hydroxysuccinimide, 1-hydroxynorbornene carboxamide or ethyl 2-hydroximino-2-cyanoacetate. In addition to minimizing O to N migration. These additives also serve as catalysts during cyclization and assist in lowering racemization. Alternatively, cyclization can be performed using the azide method, in which a reactive azide intermediate is generated from an alkyl ester via a hydrazide. Hydrazinolysis of the terminal ester necessitates the use of a t-butyl group for the protection of side chain carboxyl functions in the acylating component. This limitation can be overcome by using diphenylphosphoryl acid (DPPA), which furnishes an azide directly upon reaction with a carboxyl group. The slow reactivity of azides and the formation of isocyanates by their disproportionation restrict the usefulness of this method. The mixed anhydride method of lactam formation is widely used because of the facile removal of reaction by-products. The anhydride is formed upon reaction of the carboxylate anion with an alkyl chloroformate or pivaloyl chloride. The attack of the amino component is then guided to the carbonyl carbon of the acylating component by the electron donating effect of the alkoxy group or by the steric bulk of the pivaloyl chloride t-butyl group, which obstructs attack on the wrong carbonyl group. Mixed anhydrides with phosphoric acid derivatives have also been successfully used. Alternatively, cyclization can be accomplished using activated esters. The presence of electron withdrawing substituents on the alkoxy carbon of esters increases their susceptibility to aminolysis. The high reactivity of esters of p-nitrophenol, N-hydroxy compounds and polyhalogenated phenols has made these “active esters” useful in the synthesis of amide bonds. The last few years have witnessed the development of benzotriazolyloxytris-(dimethylamino)phosphonium hexafluorophosphonate (BOP) and its congeners as advantageous coupling reagents. Their performance is generally superior to that of the well established carbodiimide amide bond formation reactions.

Within a further embodiment, a thioether linkage may be formed between the side chain of a thiol-containing residue and an appropriately derivatized α-amino acid. By way of example, a lysine side chain can be coupled to bromoacetic acid through the carbodiimide coupling method (DCC, EDAC) and then reacted with the side chain of any of the thiol containing residues mentioned above to form a thioether linkage. In order to form dithioethers, any two thiol containing side-chains can be reacted with dibromoethane and diisopropylamine in DMF. Cyclization may also be achieved using δ₁,⊕₁′-Ditryptophan.

For longer peptide-containing agents, recombinant methods are preferred for synthesis. Within such methods, all or part of a modulating agent can be synthesized in living cells, using any of a variety of expression vectors known to those of ordinary skill in the art to be appropriate for the particular host cell. Suitable host cells may include bacteria, yeast cells, mammalian cells, insect cells, plant cells, algae and other animal cells (e.g., hybridoma, CHO, myeloma). The DNA sequences expressed in this manner may encode NK-B. NK-B sequences may be prepared based on known cDNA or genomic sequences which may be isolated by screening an appropriate library with probes designed based on such known sequences. NK-B is known from a variety of organisms including the human NK-B coding sequence. Screens may generally be performed as described in Sambrook et al., Molecular Cloning. A Laboratory Manual, Cold Spring Harbor Laboratories, Cold Spring Harbor, N.Y., 1989 (and references cited therein). Polymerase chain reaction (PCR) may also be employed, using oligonucleotide primers in methods well known in the art, to isolate nucleic acid molecules encoding all or a portion of an endogenous NK-B.

The invention further contemplates a method of generating sets of combinatorial libraries of a defined NK-B sequence. This approach is especially useful for identifying potential variant sequences (e.g. homologs) that are functional in modulating angiogenesis. Combinatorially-derived homologs can be generated which have, e.g., greater affinity, a enhanced potency relative to native NK-B peptide sequences, or intracellular half-lives different than the corresponding wild-type NK-B peptide. For example, the altered peptide can be rendered either more stable or less stable to proteolytic degradation or other cellular process which result in destruction of, or otherwise inactivation of, the peptide. Such homologs can be utilized to alter the envelope of therapeutic application by modulating the half-life of the peptide. For instance, a short half-life can give rise to more transient biological effects and can allow tighter control of peptide levels within the cell.

In one embodiment, a NK-B based peptide library can be derived by combinatorial chemistry, such as by techniques which are available in the art for generating combinatorial libraries of small organic/peptide libraries. See, for example, Blondelle et al. (1995) Trends Anal. Chem. 14:83; the Affymax U.S. Pat. Nos. 5,359,115 and 5,362,899; the Ellman U.S. Pat. No. 5,288,514; the Still et al. PCT publication WO 94/08051; Chen et al. (1994) JACS 116:2661; Kerr et al. (1993) JACS 115:252; PCT publications WO092/10092, WO93/09668 and WO91/07087; and the Lerner et al. PCT publication WO93/20242).

The combinatorial peptide library may be produced by way of a degenerate library of genes encoding a library of polypeptides which each include at least a portion of NK-B sequences. For instance, a mixture of synthetic oligonucleotides can be enzymatically ligated into gene sequences such that the degenerate set of NK-B nucleotide sequences are expressible as individual polypeptides, or alternatively, as a set of larger fusion proteins (e.g. for phage display) containing the set of NK-B-based peptide sequences therein.

There are many ways by which the gene library of potential NK-B homologs can be generated from a degenerate oligonucleotide sequence. Chemical synthesis of a degenerate gene sequence can be carried out in an automatic DNA synthesizer, and the synthetic genes then be ligated into an appropriate gene for expression. The purpose of a degenerate set of genes is to provide, in one mixture, all of the sequences encoding the desired set of potential sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp. 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

A wide range of techniques are known in the art for screening gene products of combinatorial libraries made by techniques provided above. Such techniques will be generally adaptable for rapid screening of the gene libraries generated by the combinatorial mutagenesis of NK-B derived sequences. The most widely used techniques for screening large gene libraries typically comprises cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Such illustrative assays are amenable to high throughput analysis as necessary to screen large numbers of degenerate sequences created by combinatorial mutagenesis techniques.

A peptide analog according to the present invention may comprise an internalization moiety. An internalization moiety is any moiety (such as a polypeptide, liposome or particle) that can be used to improve the ability of an agent to penetrate the lipid bilayer of the cellular plasma membrane, thus enabling the agent to readily enter the cytoplasm. In addition, an internalization moiety may also refer to a moiety capable of directing the modulating agent into the nuclear compartment. An internalization moiety may be linked via covalent attachment or a non-covalent interaction mediated by, for example, ionic bonds, hydrogen bonds, van der Waals forces and/or hydrophobic interactions, such that the internalization moiety and modulating agent remain in close proximity under physiological conditions.

In another preferred embodiment, the use of NK-B (SEQ ID NO: 1), an NK-B analog, an NK receptor agonist, or an NK receptor antagonist in the manufacture of a medicament for the treatment of an angiogenesis related condition is encompassed by the invention. In this embodiment, the medicament is used, for example, in treating rheumatoid arthritis, diabetic retinopathy, macular degeneration, atherosclerosis, psoriasis, tumor growth/metastasis, coronary artery disease, peripheral vascular disease, varicose veins or preeclampsia. In these embodiments, the NK receptor agonist or antagonist is, cycloseptide, C14TKL-1, GR 73632, hemokinin and [Sar⁹-Met(O₂)¹¹]-Substance P, senktide, [MePhe7] neurokinin B, L733060, CP99994, MK869, SDZ NKT 343, GR 82334, L-732,138, RP 67580, Spantide I, WIN51708, SR142801, SB235375, SB218795, SB222200 or combinations thereof, each commercially available.

In the generation of modulating agents including NK-B peptide analogs described herein, it may be necessary to include unstructured linkers in order to ensure proper folding of the peptide, and prevent steric or other interference of the respective molecule. Many synthetic and natural linkers are known in the art and can be adapted for use in the present invention. In general, spacers may be amino acid residues (e.g., amino hexanoic acid) or peptides, or may be other bi- or multi-functional compounds that can be covalently linked to at least two peptide sequences. Covalent linkage may be achieved via direct condensation or other well known techniques.

According to one aspect of this invention, modulating agents according to the present invention may be administered directly to target cells of patients in need thereof. Direct delivery of such therapeutics may be facilitated by formulation of the composition in any pharmaceutically acceptable dosage form, e.g., for delivery orally, intratumorally, peritumorally, interlesionally, intravenously, intramuscularly, subcutaneously, periolesionally, or topical routes, to exert local therapeutic effects.

Topical administration of the therapeutic is advantageous since it allows localized concentration at the site of administration with minimal systemic adsorption. This simplifies the delivery strategy of the agent to the disease site and reduces the extent of toxicological characterization. Furthermore, the amount of material to be applied is far less than that required for other administration routes.

In one embodiment, the membrane barrier can be overcome by utilizing an internalization moiety comprising lipid formulations closely resembling the lipid composition of natural cell membranes. In particular, the subject peptides, analogs, or peptidomimetics are encapsulated in liposomes to form pharmaceutical preparations suitable for administration to living cells. Yarosh, U.S. Pat. No. 5,190,762 demonstrates that proteins can be delivered across the outer skin layer and into living cells, without receptor binding, by liposome encapsulation. These lipids are able to fuse with the cell membranes on contact, and in the process, the associated peptides, analogs, or peptidomimetics are delivered intracellularly. Lipid complexes can not only facilitate intracellular transfers by fusing with cell membranes but also by overcoming charge repulsions between the cell membrane and the molecule to be inserted. The lipids of the formulations comprise an amphipathic lipid, such as the phospholipids of cell membranes, and form hollow lipid vesicles, or liposomes, in aqueous systems. This property can be used to entrap peptides, analogs, or peptidomimetics within the liposomes.

Liposomes offer several advantages. They are non-toxic and biodegradable in composition; they display long circulation half-lives; and recognition molecules can be readily attached to their surface for targeting to tissues. Finally, cost effective manufacture of liposome-based pharmaceuticals, either in a liquid suspension or lyophilized product, has demonstrated the viability of this technology as an acceptable drug delivery system.

Liposomes have been described in the art as in vivo delivery vehicles. The structure of various types of lipid aggregates varies, depending on composition and method of forming the aggregate. Such aggregates include liposomes, unilamellar vesicles, multilamellar vesicles, micelles and the like, having particle sizes in the nanometer to micrometer range. Methods of making lipid aggregates are by now well-known in the art. For example, the liposomes may be made from natural and synthetic phospholipids, glycolipids, and other lipids and lipid congeners; cholesterol, cholesterol derivatives and other cholesterol congeners; charged species which impart a net charge to the membrane; reactive species which can react after liposome formation to link additional molecules to the liposome membrane; and other lipid soluble compounds which have chemical or biological activity.

In another embodiment, the present invention relates to gene therapy constructs containing a nucleic acid encoding an NK-B peptide of the present invention, operably linked to at least one transcriptional regulatory sequence. Such constructs may encode a nuclear localization signal, either from native NK-B or from another source, which acts to direct the peptide to the nuclear compartment. The gene constructs of the present invention are formulated to be used as a part of a gene therapy protocol to deliver the subject therapeutic protein to a target cell in an animal.

Any of the methods known to the art for the insertion of DNA fragments into a vector may be used to construct expression vectors consisting of appropriate transcriptional/translational control signals and the desired NK-B peptide-encoding nucleotide sequence. See, for example, Maniatis T., Fritsch E. F., and Sambrook J. (1989): Molecular Cloning (A Laboratory Manual), Cold Spring Harbor Laboratory, Cold Spring Harbor, N.Y.; and Ausubel F. M., Brent R., Kingston R. E., Moore, D. D., Seidman J. G., Smith J. A., and Struhl K. (1992): Current Protocols in Molecular Biology, John Wiley & Sons, New York. These methods may include in vitro DNA recombinant and synthetic techniques and in vivo genetic recombination. Expression of a nucleic acid sequence encoding a peptide may be regulated by a second nucleic acid sequence so that the peptide is expressed in a host infected or transfected with the recombinant DNA molecule. For example, expression of a NK-B peptide may be controlled by any promoter/enhancer element known in the art. The promoter activation may be tissue specific or inducible by a metabolic product or administered substance.

Promoters/enhancers which may be used to control the expression of the NK-B peptide in vivo include, but are not limited to, the native NK-B promoter, the cytomegalovirus (CMV) promoter/enhancer (Karasuyama et al., 1989, J. Exp. Med., 169:13), the human β-actin promoter (Gunning et al. (1987) PNAS 84:4831-4835), the glucocorticoid-inducible promoter present in the mouse mammary tumor virus long terminal repeat (MMTV LTR) (Klessig et al. (1984) Mol. Cell Biol. 4:1354-1362), the long terminal repeat sequences of Moloney murine leukemia virus (MuLV LTR) (Weiss et al. (1985) RNA Tumor Viruses, Cold Spring Harbor Laboratory, Cold Spring Harbor, N.Y.), the SV40 early or late region promoter (Bemoist et al. (1981) Nature 290:304-310; Templeton et al. (1984) Mol. Cell Biol, 4:817; and Sprague et al. (1983) J. Virol., 45:773), the promoter contained in the 3′ long terminal repeat of Rous sarcoma virus (RSV) (Yamamoto et al., 1980, Cell, 22:787-797), the herpes simplex virus (HSV) thymidine kinase promoter/enhancer (Wagner et al. (1981) PNAS 82:3567-71), and the herpes simplex virus LAT promoter (Wolfe et al. (1992) Nature Genetics, 1:379-384), and Keratin gene promoters, such as Keratin 14.

Expression constructs of the subject NK-B peptides may be administered in any biologically effective carrier, e.g. any formulation or composition capable of effectively delivering the recombinant gene to cells in vivo. Approaches include insertion of the NK-B peptide coding sequence in viral vectors including recombinant retroviruses, adenovirus, adeno-associated virus, and herpes simplex virus-1, or recombinant eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct or CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically.

A preferred approach for in vivo introduction of nucleic acid into a cell is by use of a viral vector containing nucleic acid encoding the particular NK-B peptide possessing angiogenesis modulating activity. Infection of cells with a viral vector has the advantage that a large proportion of the targeted cells can receive the nucleic acid. Additionally, molecules encoded within the viral vector, e.g., the recombinant NK-B peptide, are expressed efficiently in cells which have taken up viral vector nucleic acid.

In addition to viral transfer methods, such as those illustrated above, non-viral methods can also be employed to cause expression of a NK-B peptide in the tissue of an animal. Most nonviral methods of gene transfer rely on normal mechanisms used by mammalian cells for the uptake and intracellular transport of macromolecules. In preferred embodiments, non-viral gene delivery systems of the present invention rely on endocytic pathways for the uptake of the construct encoding the NK-B polypeptides by the targeted cell. Exemplary gene delivery systems of this type include liposomal derived systems, poly-lysine lysine conjugates, and artificial viral envelopes.

In clinical settings, the gene delivery systems for the therapeutic NK-B peptide coding sequence can be introduced into a patient by any of a number of methods, each of which is familiar in the art. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or “gene gun” techniques. In preferred embodiments, the gene therapy construct of the present invention is applied topically to target cells of the skin or mucosal tissue. A NK-B peptide gene construct can, in one embodiment, be delivered in a gene therapy construct by electroporation using techniques described, for example, by Dev et al. ((1994) Cancer Treat Rev 20:105-115).

The pharmaceutical preparation of the gene therapy construct can consist essentially of the gene delivery system in an acceptable diluent, or can comprise a slow release matrix in which the gene delivery vehicle is imbedded. Alternatively, where the complete gene delivery system can be produced intact from recombinant cells, e.g. retroviral vectors, the pharmaceutical preparation can comprise one or more cells which produce the gene delivery system.

The ability of analogs according to the invention to modulate angiogenesis may generally be evaluated using any suitable assay known to those of ordinary skill in the art. An illustrative in vitro angiogenesis assay is described below in the Examples section. Peptide analogs useful in methods according to the present invention are identified as those agents capable of providing a statistically meaningful difference in angiogenesis, namely blood vessel formation, in comparison to controls.

An peptide analog or peptidomimetic according to the present invention may, but need not, be linked to one or more additional molecules. Although molecules as described herein may preferentially bind to specific tissues or cells, and thus may be sufficient to target a desired site in vivo, it may be beneficial for certain applications to include an additional targeting agent. Accordingly, a targeting agent may be associated with an agent to facilitate targeting to one or more specific tissues. As used herein, a “targeting agent” may be any substance (such as a compound or cell) that, when associated with a modulating agent enhances the transport of the modulating agent to a target tissue, thereby increasing the local concentration of the modulating agent.

Targeting agents include antibodies or fragments thereof, receptors, ligands and other molecules that bind to cells of, or in the vicinity of, the target tissue. Known targeting agents include serum hormones, antibodies against cell surface antigens, lectins, adhesion molecules, tumor cell surface binding ligands, steroids, cholesterol, lymphokines, fibrinolytic enzymes and those drugs and proteins that bind to a desired target site. Among the many monoclonal antibodies that may serve as targeting agents are anti-TAC, or other interleukin-2 receptor antibodies; 9.2.27 and NR-ML-05, reactive with the 250 kilodalton human melanoma-associated proteoglycan; and NR-LU-10, reactive with a pancarcinoma glycoprotein. An antibody targeting agent may be an intact (whole) molecule, a fragment thereof, or a functional equivalent thereof. Examples of antibody fragments are F(ab′)2, -Fab′, Fab and F[v] fragments, which may be produced by conventional methods or by genetic or protein engineering. Linkage is generally covalent and may be achieved by, for example, direct condensation or other reactions, or by way of bi- or multi-functional linkers. Within other embodiments, it may also be possible to target a polynucleotide encoding a modulating agent to a target tissue, thereby increasing the local concentration of modulating agent. Such targeting may be achieved using well known techniques, including retroviral and adenoviral infection, as described above.

Within certain aspects of the present invention, one or more modulating agents as described herein may be present within a pharmaceutical composition. A pharmaceutical composition comprises one or more modulating agents in combination with one or more pharmaceutically or physiologically acceptable carriers, diluents or excipients. Such compositions may comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Within yet other embodiments, compositions of the present invention may be formulated as a lyophilizate. One or more modulating agents (alone or in combination with a targeting agent and/or drug) may, but need not, be encapsulated within liposomes using well known technology. Compositions of the present invention may be formulated for any appropriate manner of administration, including for example, topical, oral, nasal, intravenous, intracranial, intraperitoneal, subcutaneous, or intramuscular administration.

A pharmaceutical composition may also, or alternatively, contain one or more drugs, which may be linked to a modulating agent or may be free within the composition. Virtually any drug may be administered in combination with a modulating agent as described herein, for a variety of purposes as described below. Examples of types of drugs that may be administered with a modulating agent include analgesics, anesthetics, antianginals, antifingals, antibiotics, anticancer drugs (e.g., taxol or mitomycin C), antiinflammatories (e.g., ibuprofen and indomethacin), antihelmintics, antidepressants, antidotes, antiemetics, antihistamines, antihypertensives, antimalarials, antimicrotubule agents (e.g., colchicine or vinca alkaloids), antimigraine agents, antimicrobials, antiphsychotics, antipyretics, antiseptics, anti-signaling agents (e.g., protein kinase C inhibitors or inhibitors of intracellular calcium mobilization), antiarthritics, antithrombin agents, antituberculotics, antitussives, antivirals, appetite suppressants, cardioactive drugs, chemical dependency drugs, cathartics, chemotherapeutic agents, coronary, cerebral or peripheral vasodilators, contraceptive agents, depressants, diuretics, expectorants, growth factors, hormonal agents, hypnotics, immunosuppression agents, narcotic antagonists, parasympathomimetics, sedatives, stimulants, sympathomimetics, toxins (e.g., cholera toxin), tranquilizers and urinary antiinfectives.

The compositions described herein may be administered as part of a sustained release formulation (i.e., a formulation such as a capsule or sponge that effects. a slow release of modulating agent following administration). Such formulations may generally be prepared using well known technology and administered by, for example, oral, rectal or subcutaneous implantation, or by implantation at the desired target site. Sustained-release formulations may contain a modulating agent dispersed in a carrier matrix and/or contained within a reservoir surrounded by a rate controlling membrane (see, e.g., European Patent Application 710,491 A). Carriers for use within such formulations are biocompatible, and may also be biodegradable; preferably the formulation provides a relatively constant level of modulating agent release. The amount of modulating agent contained within a sustained release formulation depends upon the site of implantation, the rate and expected duration of release and the nature of the condition to be treated or prevented.

Pharmaceutical compositions of the present invention may be administered in a manner appropriate to the disease to be treated (or prevented). Appropriate dosages and a suitable duration and frequency of administration will be determined by such factors as the condition of the patient, the type and severity of the patient's disease and the method of administration. In general, an appropriate dosage and treatment regimen provides the modulating agent(s) in an amount sufficient to provide therapeutic and/or prophylactic benefit.

By the term “disorder”, it is meant an abnormal physical. As used herein, “disorders” include, but are not limited to, diseases. By the term “symptom”, it is meant subject evidence of disease or something that indicates the presence of a bodily disorder, such as, but not limited to, a disease.

The phrase “inhibitory amount”, as used herein, means an amount of an agent (a compound or composition) which is sufficient to reduce the level or activity of angiogenesis to a statistically significant lesser value as compared to when the agent is not present.

### III. EXAMPLES

A. General: Protocols and Reagents

Neurokinin B (NK-B), NK1 receptor-selective antagonist L733060 [(2S,3S)-3-[93,5-bis(trifluoro methyl)phenyl)methoxy]-2-phenylpiperidine hydrochloride], NK3 receptor-selective antagonist SB222200 [(S)-3 methyl-2-phenyl-N-(1-phenylpropyl)-4-quinilinecarboxamide], and forskolin were purchased from Sigma (St. Louis, Mo.). U46619 and IBMX were purchased from Calbiochem (La Jolla, Calif.) and were solubilized in DMSO. U46619 was diluted in ethanol. The final concentrations of NK-B, IBMX and forskolin were 100 μM, 100 μM, and 10 μM respectively. The final concentrations of DMSO and ethanol did not exceed 1%. Recombinant mouse VEGF164 and bovine FGF2 from R & D systems (Minneapolis, Minn.) were reconstituted in PBS containing 1% BSA.

### Example 1

### Synthesis of Mutant NK-B

An NK-B mutant peptide was produced via microwave-assisted solid-phase peptide synthesis (Murray and Gellman, 2005). Fmoc-amino acids (Novabiochem, San Diego, Calif.) were activated with HBTU/HOBt in DMF and coupled using microwave irradiation (600 W maximum power, 70° C., ramp 2 min, hold 2 min, CEM MARs multimode microwave). Removal of the Fmoc protecting group was accomplished by treatment with 20% piperidine in DMF with microwave irradiation (600 W maximum power, 80° C., ramp 2 min, hold 2 min). Following cleavage from the solid support (NovaSyn TGR resin) with TFA, the crude peptide mixture was purified by reverse phase HPLC and structurally validated by MALDI-TOF MS.

### Example 2

### Cell Lines and Cell Culture

Yolk sac endothelial cells (YSECs), HUVECs and human aortic endothelial cells (HAECs) were maintained in M200 medium (Cascade Biologics), and HMVECs were maintained in M131 medium (Cascade Biologics). The culture medium was supplemented with low serum growth supplement (LSGS) (Cascade Biologics) containing fetal bovine serum (2%), hydrocortisone. (1 μg/ml), human epidermal growth factor (10 ng/ml), fibroblast growth factor-2 (3 ng/ml) and heparin (10 μg/ml). Human endothelial cells were used between passages 2 and 8.

Mouse yolk sac endothelial cells (YSECs) were derived from a hypervascular transgenic mouse expressing the fps/fes protooncogene (Lu et al., 1996). YSECs exhibit a normal endothelial phenotype and are not tumorigenic. HUVECs, HAECs, and HMVECs were from Cascade Biologics (Portland, Oreg.). Cells were maintained as described above.

### Example 3

### Cell Proliferation Assay

YSECs were seeded in 6-well plates at 10,000 cells/ml/well and allowed to adhere. After 5 h, fresh medium containing vehicle, NK-B, the phosphodiesterase inhibitor 3-Isobutyl-1-methylxanthine (IBMX) (A.G. Scientific, Inc., San Diego, Calif.), or NK-B/IBMX was added. At 24 h intervals, cells were trypsinized, and viable cells were scored.

### Example 4

### Vascular Network Assembly Assay

Vascular network assembly was assessed by measuring the formation of capillary-like structures by endothelial cells on Matrigel (BD Biosciences, San Jose, Calif.). Matrigel was diluted 1:1 with supplement-free M200 medium, poured in 24-well plates, and allowed to solidify at 37° C. Subconfluent endothelial cells were harvested and preincubated under different experimental conditions (YSECs, 1 h; HUVECs, HAECs, and human microvascular endothelial cells (HMVECs), 2 h in supplement-free M200 medium in microfuge tubes. An equal volume of supplemented medium containing the indicated reagents was added. Cells were plated on Matrigel (1.5×10⁵ cells/well) and incubated at 37° C. Vascular network assembly was measured as a function of time, and digital pictures were captured.

### Example 5

### Cell Migration Analysis by Time-Lapse Video Microscopy

Subconfluent YSECs were harvested and pretreated with vehicle, IBMX, IBMX/NK-B, or IBMX/forskolin for 1 h in supplement-free M200 medium; 5-10×10⁴ cells were plated per well in a 12-well Matrigel-coated plate, and an equal volume of supplemented medium with or without various reagents was added. NK receptor inhibitors were added 30 min before adding NK-B. Cells were allowed to adhere for 30 min, and time-lapse images were acquired at 37° C. with a TE300 Nikon inverted microscope with a 20× objective (Garden City, N.Y.), a Photometrics CoolSnap fx CCD camera (Tucson, Ariz.), and a cube temperature controller (Life Imaging Services, Reinach, Switzerland). Images were captured with E-See Inovision Software (Inovision, Raleigh, N.C.) (one image per minute for 120 min). Cell migration was quantitated by scoring the number of cells migrating in a field and expressed as a percentage of the total cells.

### Example 6

### cAMP Assay

Cells were cultured in 6-well plates (2×10⁵ cells/well), washed with supplement-free M200 medium and treated with indicated reagents at 37° C. The reactions were terminated by aspiration of the medium. Cells were washed with PBS and lysed in 500 μl of 0.1 N HCl containing 0.5% Triton X-100. cAMP was assayed in cell lysates (100 μl) using a direct cAMP enzyme-linked immunosorbent assay (Assay Designs, Inc., Ann Arbor, Mich.).

### Example 7

### Chick Chorioallantoic Membrane Assay

Fertilized chicken eggs were incubated at 39° C. with 90% humidity. On embryonic day 7, a small hole was drilled at the end of the egg that contains the air sac to lower the embryo. A second hole was made above the chorioallantoic membrane (CAM), and 100 μl of a 1.5% solution of methylcellulose (Stem Cell Technologies, Vancouver, BC) containing either FGF2 (500 ng) or VEGF (200 ng) and vehicle, IBMX (100 μg), NK-B (40 μg)/IBMX, or mutant NK-B (40 μg)/IBMX were applied to the CAM. A solution of penicillin/streptomycin was added, and the eggs were sealed with transparent tape. Vasculature was analyzed after 48 h, and digital images were captured at 40× magnification.

### Example 8

### Calcium Imaging

Cells were incubated for ˜30 min in 4 μM fluo-4/AM (Molecular Probes, Carlsbad, Calif.) in supplement-free M200 medium at 37° C., trypsinized, plated on Matrigel with indicated reagents, and allowed to adhere for 10 min. Time-lapse video microscopy with an Olympus AX70 laser scanning confocal microscope was used to measure intracellular Ca⁺² as described previously (Robles et al., 2003). An argon laser at 488 nm was used to excite Fluo-4, and the emitted light was detected at wavelengths>510 nm. Images were acquired at 15 s intervals and analyzed using Image J (Image Processing and Analysis in JAVA, Nat'l Inst. Health). When indicated, the fluorescence intensity in the region of interest (F) was normalized by dividing the Ca+2 signal by the fluorescence intensity of the resting cell at time 0 (F₀).

### Example 9

### RNA Interference

To generate stably expressing siRNAs, oligonucleotides were designed according to the criteria specified by Dharmacon and Oligoengine and cloned into the BglII/HindIII sites of pSUPER Puro (Oligoengine, Seattle, Wash.). The NK1 target sequence, 5′-CAACAGGACTTACGAGAAA-3′ (SEQ ID NO: 2), corresponded to nucleotides 1480-1497 of the mouse NK1 cDNA; the NK3 target sequence, 5′-AGATTTCGTGCAGGCTTCA-3′ (SEQ ID NO: 3), corresponded to nucleotides 1044-1061 of the mouse NK3 cDNA. The empty vector was used as a negative control. YSECs were transfected using Lipofectin (Invitrogen, Carlsbad, Calif.), and positive clones were selected with puromycin (2 μg/ml).

### Example 10

### Gene Expression Analysis

Real-time RT-PCR was conducted Total RNA was purified with Trizol (Invitrogen). cDNA was prepared by annealing RNA (1 μg) with 250 ng of a 1:5 mixture of random and oligo(dT) primers (Promega) at 68° C. for 10 min, followed by incubation with Moloney Murine Leukemia Virus reverse transcriptase (Invitrogen) with 10 mM dithiothreitol, RNasin (Promega), and 0.5 mM dNTPs at 42° C. for 1 h. Reactions were diluted to a final volume of 100 μl and subjected to heat-inactivation at 98° C. for 10 min. Reactions (25 μl) contained 2.5 μl of cDNA, 12.5 μl of SYBR Green Master Mix (Applied Biosystems), and appropriate primers. Product accumulation was monitored by SYBR Green fluorescence. Control reactions lacking RT yielded little or no signals. The relative expression levels were determined from a standard curve of serial dilutions of cDNA samples from untreated control cells, and measurements were conducted under conditions of linearity.

Real time RT-PCR primers were designed using PRIMER EXPRESS 1.0 (PE Applied Biosystems) to amplify 50-150 bp amplicons and were based on GenBank Ensembl sequences. Sequences are provided in TABLE 1, below.

Forward and reverse primers for RT-PCR analysis are shown in TABLE I.

TABLE 1

SEQ

| | | |
|---|---|---|
| Forward Primer | Reverse Primer | ID |

| | | | |
|---|---|---|---|
| GENE | (5′-3′) | (3′-5′) | NO |

| | | | |
|---|---|---|---|
| Mouse | tgcccccatgtttgtgatg | tgtggtcatgagcccttc | 4/5 |
| GAPDH | | c | |
| Mouse | ttgtcatctgggtcctggc | gtagatcagcacagtcac | 6/7 |
| NK1 | t | acagatgt | |
| Mouse | ccagaaggtcccaagcaac | tgggaaacagtacaccag | 8/9 |
| NK3 | at | gatg | |
| Mouse | tgaaggaagatggacggct | tccagtcgttcaaagaag | 10/11 |
| FGF-2 | g | aaacac | |
| Mouse | tggactgagaccaagccca | cccgcctccttgctttta | 12/13 |
| Flt-1 | ct | | |
| Mouse | ttggcaaatacaacccttc | cctttcctcagaatcacg | 14/15 |
| Flk-1 | aga | ctg | |
| Mouse | ttggaggctacaaggttcg | tcagaaggcaccacagaa | 16/17 |
| FGFR-1 | c | tcc | |
| Human | attggtggagatgatctct | gccagtgtcaattatatc | 18/19 |
| HPRT | caacttt | ttccacaa | |
| Human | agtcgtgtgcatgatcgaa | tgcatagccaatcaccag | 20/21 |
| NK1 | tg | ca | |
| Human | tactccaccgtcaccatgg | tgtaggctacaaacatca | 22/23 |
| NK2 | ac | ccgc | |
| Human | ccgcttccagaacttcttt | gacagtctgggtttcaag | 24/25 |
| NK3 | cc | ggat | |
| Human | ctgccactctaattgtcaa | aaacgatgacacggcctt | 26/27 |
| Flt-1 | tgtgaa | tt | |
| Human | gagggagaagtccctcagt | ccttatacagatcttcag | 28/29 |
| Flk-1 | gatgtga | gagcttcc | |

### Example 11

### 2D Gel Electrophoresis and Mass Spectrometry

YSECs were treated with IBMX or NK-B/IBMX for 1 h in supplement-free M200 medium, followed by 10 min. in supplemented medium. Cells were processed and proteins analyzed as described above in Example 2.

Cells were harvested, washed twice with ice-cold PBS, and lysed in lysis buffer containing 10 mM Tris (pH 7.4), 0.3% SDS, 100 μg/mL Ribonuclease A (Sigma), 200 μg/mL DNase I (Sigma), 20 μg/mL leupeptin (Sigma), 6 μg/mL E-64 (Sigma), 1 mM EDTA (Calbiochem), and 100 μg/mL benzamidine (Sigma). Cells were incubated on ice for 30 min, an equal volume of loading buffer containing 60 mM Tris (pH 6.8), 5% SDS, 10% glycerol was added, and samples were boiled for 5 min. 2D gel electrophoresis was conducted using pH 3.5-10 ampholines, and proteins were detected by Coomassie Blue staining. MALDI mass spectrometry was conducted by the Columbia University protein core facility

### Example 12

### Antibodies and Immunoblot Analysis

The following antibodies were used for Western blots: Anti-phospho-FAK (Tyr-397) (Biosource, 44-624G); anti-FAK, anti-phospho-Akt (Ser 473), anti-Akt, anti-phospho-p44/42 MAP kinase (Thr 202/Tyr 204), anti-p44/42 MAP kinase antibodies (Cell Signaling Technology); anti-calreticulin (Upstate, 06-661); anti-α-tubulin (Calbiochem); anti Flt-1 (Santa Cruz Biotech, C-17); anti-Flk-1 (Cell Signaling Technologies, 2479). Total protein was prepared by boiling cells for 5 min in 50 mM Tris (pH 6.8), 100 mM DTT, 2% SDS, 0.1% bromophenol blue, and 10% glycerol (1×10⁷ cells/ml). Extracts were resolved on SDS polyacrylamide gels, transferred to Immobilon P membranes (Millipore, Billerica, Mass.), and analyzed by Western blotting. Proteins were detected by chemiluminescence using ECL-Plus (Amersham Biosciences Piscataway, N.J.).

### Example 13

### Recombinant Calreticulin and Vasostatin Production

For construction of the glutathione S transferase (GST)-calreticulin and -vasostatin fusion constructs, the coding regions for calreticulin and vasostatin were cloned as C-terminal translational fusions with the GST gene for expression in E coli. Purification of GST-calreticulin was achieved by lysis of the bacteria, followed by sonication, centrifugation, adjusting the pH of supernatants to pH 7.0, and mixing with pre-equilibrated Glutathione Sepharose 4B (Amersham Biosciences) in PBS containing 1.0% Triton X-100. After a 30 min incubation, beads were washed, bound protein was cleaved with factor Xa (Novagen, San Diego, Calif.), and liberated protein was separated from immobilized GST via centrifugation.

B. Promotion of Angiogenesis

### Example 14

### NK-B Reversibly Opposes Endothelial Cell Vascular Network Assembly

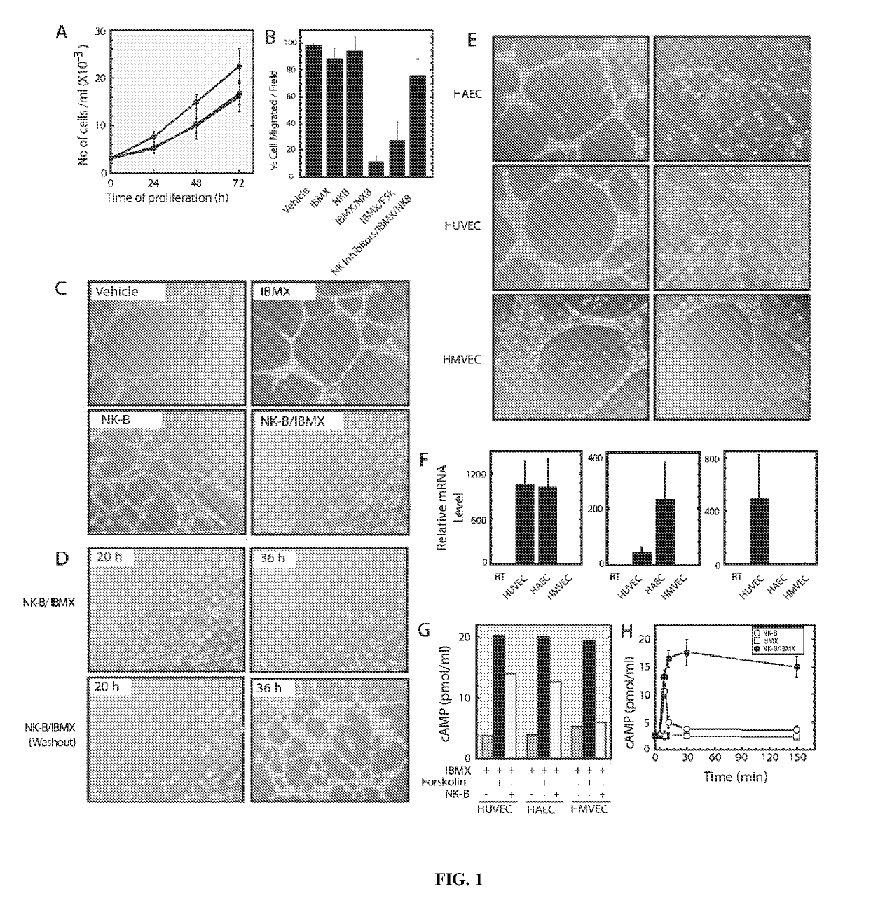

Based on previous research the inventors began investigating the effect of NK-B on endothelial cells. Therefore, the effect of NK-B was tested on various endothelial cell types. To confirm their results several endothelial cell types were used including HUVECs, HAECs and YSECs. To further elucidate the effect of NK-B, IBMX an agent know for elevating cAMP levels was also used either alone or in combination with NK-B. FIGS. 1A-H shows the effects of a first set of these experiments. FIG. 1A shows the results of a YSEC proliferation assay. YSECs (10⁴ cells/well, 6-well plate) were plated with vehicle, IBMX, NK-B, or NK-B/IBMX, and the number of viable cells/well was counted after 24, 48, and 72 h (mean+/−S.E., 3 independent experiments). FIG. 1B, YSEC motility assay. YSECs were treated with the indicated reagents in supplement-free M200 medium for 1 h and plated on Matrigel with an equal volume of Low Serum Growth Supplement (LSGS)-containing M200 medium (supplemented medium) containing the same reagents. Cells were allowed to adhere for 20 min, and cell migration was then monitored using time-lapse video microscopy for 2 h. Migrating YSECs were counted and expressed as a percent of the total number of cells (mean+/−S.E., 4 independent experiments). FIG. 1C, YSEC vascular network assembly assay. Cells were treated with vehicle, IBMX, NK-B, or NK-B/IBMX for 1 h in supplement-free M200 medium, plated on Matrigel containing supplemented medium, and incubated for 20 h at 37° C. FIG. 1D, reversibility of vascular network assembly blockade. YSECs were treated with NK-B/IBMX and plated on Matrigel for 20 h. The NK-B/IBMX-containing medium was replaced with IBMX-containing medium (bottom panel), and cells were cultured for 16 h. FIG. 1E, HAECs, HUVECs and HMVECs were treated with IBMX or NK-B/IBMX for 2 h in supplement-free M200 medium, plated on Matrigel containing supplemented medium, and incubated for 12 h. FIG. 1F, quantitative real-time RT-PCR analysis of NK1, NK2, and NK3 receptor mRNA levels in HUVECs, HAECs, and HMVECs. The relative mRNA levels were normalized by HPRT mRNA levels. The plots depict the mRNA ratios in which the ratios obtained for the −RT condition were designated as 1 (mean+/−S.E., three independent experiments). FIG. 1G HUVECs, HAECs, HMVECs were treated with IBMX with or without forskolin or NK-B for 1 h. cAMP was quantitated in cell lysates (mean+/−S.E., 3 independent experiments). FIG. 1H YSECs were treated with NK-B or NK-B/IBMX in supplement-free M200 medium, and cAMP was assayed at different times (mean+/−S.E., 3 independent experiments). These data indicate that, compared to vehicle and IBMX, NK-B inhibits the formation of the endothelial tubular network. In addition, while cells treated with IBMX alone showed little difference from those treated with vehicle, when given together IBMX potentiated the effect of NK-B in inhibiting endothelial network formation. Further, this effect is reversible as indicated by the washout data shown in FIG. 1D.

### Example 15

### NK-B is Anti-Angiogenic

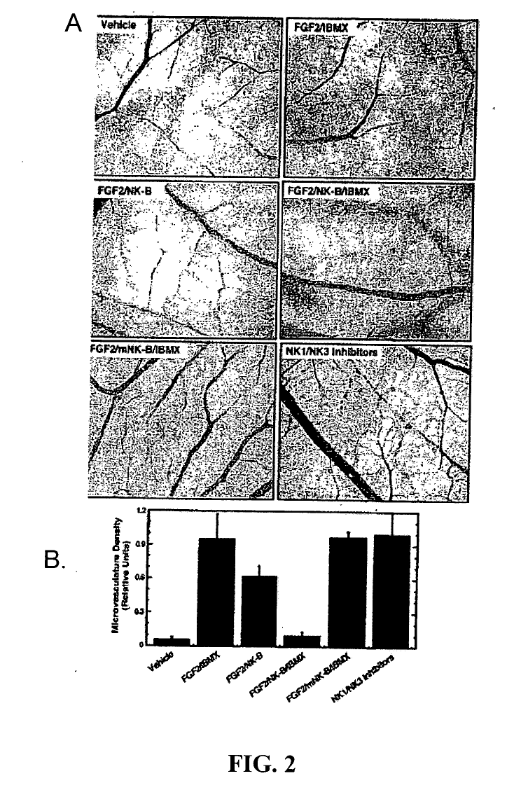

The inventors next used the chick embryo to test whether the in vitro effect of NK-B on endothelial cell network assembly held true for in vivo network assembly. Thus, eggs were prepared that allowed the observation of the chick chorioallantoic membrane (CAM) during treatment with the various compounds. The chick embryo were treated as follows: FIG. 2A, methylcellulose containing vehicle; FIG. 2B, FGF2 (500 ng)/IBMX (100 μg); FIG. 2C, FGF2/NK-B (40 μg); FIG. 2D FGF2/NK-B (40 μg) with IBMX; FIG. 2E, FGF2/mNK-B/IBMX (mNK-B, inactive mutant of NK-B); FIG. 2F, a combination of NK1-(L733060, 5 μM) and NK3-(SB222200, 2 μM) selective inhibitors; each applied to the CAM of day 7 chicken embryos. After 48 h, blood vessels were analyzed, digital images were captured at 40× magnification, and representative images are shown (15-20 eggs were analyzed per condition, 3-4 independent experiments). FIG. 2G is a histogram that depicts the relative microvasculature density in each condition FIGS. 2A-F determined by overlaying a grid on the Adobe Illustrator images. These data clearly illustrated that NK-B given in combination with angiogenic agents such as FGF2 and FGF2/IBMX decreases the microvascular density. Further, the specificity of the NK-B effect is shown by the increased vascular density of the embryo when the NK-B signal is blocked using various NK-B receptor inhibitors. Thus, NK-B is anti-angiogenic and disruption of endogenous neurokinin signaling is angiogenic in vivo in the chicken chorioallantoic membrane.

### Example 16

### NK Receptor Requirement for NK-B-Mediated Abrogation of Vascular Network Assembly

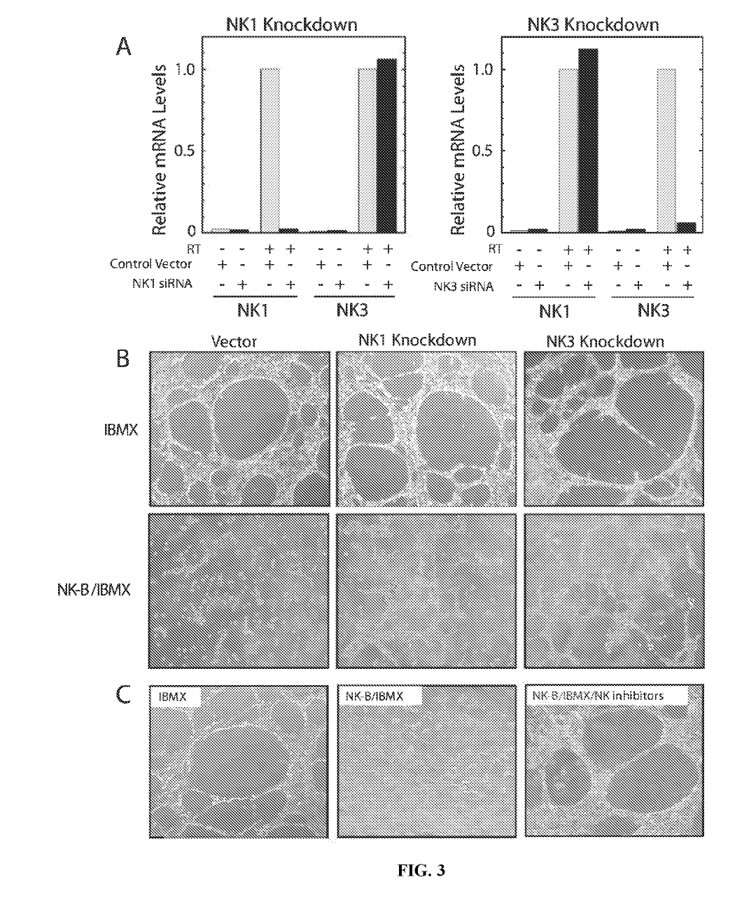

The experiment described in Example 15 indicates that the anti-angiogenic effect of NK-B may be mediated by more than one NK-B receptor. Therefore, the inventors designed further experiments to elucidate the NK receptor requirement for NK-B-mediated abrogation of vascular network assembly. For these experiments, vectors containing small interfering RNA (siRNA) for the NK-B receptors NK1 and NK3 were prepared as described in Example 9. FIG. 3A shows the results of quantitative RT-PCR analysis of NK1 and NK3 mRNA in YSEC clonal lines stably expressing NK1 (left panel) and NK3 (right panel) siRNA molecules (RT, reverse transcriptase). The siRNA essentially inhibit expression of each specific receptor. FIG. 3B, vascular network assembly assay with control (left panel), NK1 knockdown (middle panels) and NK3 knockdown (right panel). Control and siRNA-expressing YSECs were treated with IBMX or NK-B/IBMX and incubated on Matrigel for 20 h at 37° C. Knocking-down either NK1 or NK3 reduced the capacity of NK-B/IBMX to abrogate vascular network assembly. FIG. 3C, YSECs were treated with IBMX or NK-B/IBMX with or without NK1-(L733060) and NK3-(SB222200) selective inhibitors for 15 min, followed by NK-B/IBMX, and then plated on Matrigel to assess vascular network assembly. These data confirm that NK-B mediates at least some of its effect on vascular network assembly via NK receptors.

### Example 17

### NK-B Signaling Circuitry

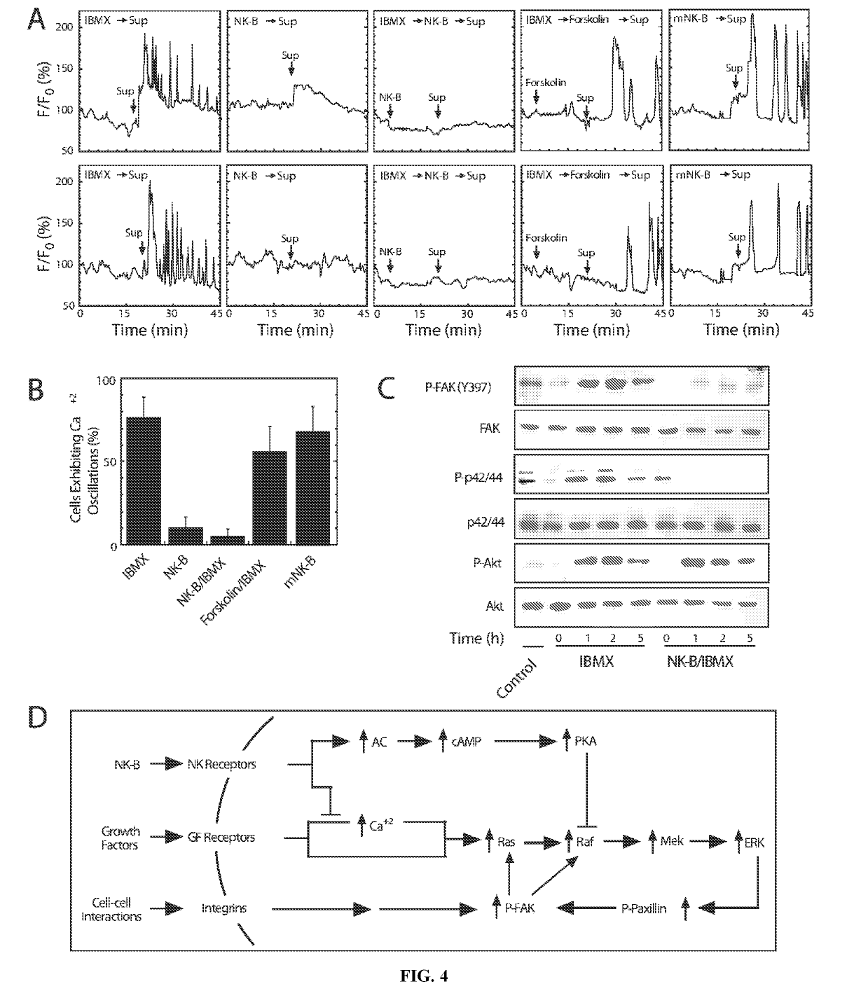

In light of the foregoing experiments the inventors designed further experiments to more clearly elucidate the NK-B signaling circuitry. Thus, the investigators designed experiments to investigate the effect of various substances on calcium oscillations. FIG. 4A shows real-time measurements of Ca⁺² oscillations, F/Fo indicates % increase in fluorescence intensity. YSECs were loaded with Fluo-4 AM in supplement-free M200 medium and plated on Matrigel under different experimental conditions. Intracellular Ca⁺² was monitored every 15 s. Ca⁺² oscillation patterns are plotted as a percentage of fluorescence intensity at time 0 (F₀), of two representative cells (2 independent experiments per condition) (Sup, supplemented M200 medium). FIG. 4B is a graph of quantitative analysis of Ca⁺² oscillation patterns, percentage of cells exhibiting Ca⁺² oscillations (mean+/−SE, 3 independent experiments). FIG. 4C, is a Western blot showing the effects of YSECs treated with IBMX or NK-B/IBMX in supplement-free medium for 1 h. Cells were plated on Matrigel containing supplemented medium for 0.5, 1, 2, and 5 h, and cell lysates were analyzed for phosphorylated and nonphosphorylated forms of FAK kinase, p42/44 MAPK, and Akt. Cells continuously grown in the presence of supplemented medium were used as a control. These data were compiled to generate the signaling crosstalk pathway illustrated in FIG. 4D. As shown, NK-B activates NK receptors, which are coupled to adenylyl cyclase (AC), thereby increasing cAMP levels and activating protein kinase A (PKA). PKA inactivates Raf via direct phosphorylation. NK-B also abrogates growth factor (GF)-dependent Ca⁺² oscillations. As Ca⁺² oscillations activate Ras, decreased Ca⁺² oscillations represent a second mechanism whereby NK-B antagonizes the Ras-ERK pathway, which was measured as reduced phosphorylation of p42/p44 in FIG. 4C. NK-B also decreased FAK autophosphorylation on Y397, which can be explained by the NK-B antagonism of the Ras-ERK pathway.

### Example 18

### NK-B Downregulates Type I and Type II VEGF Receptors

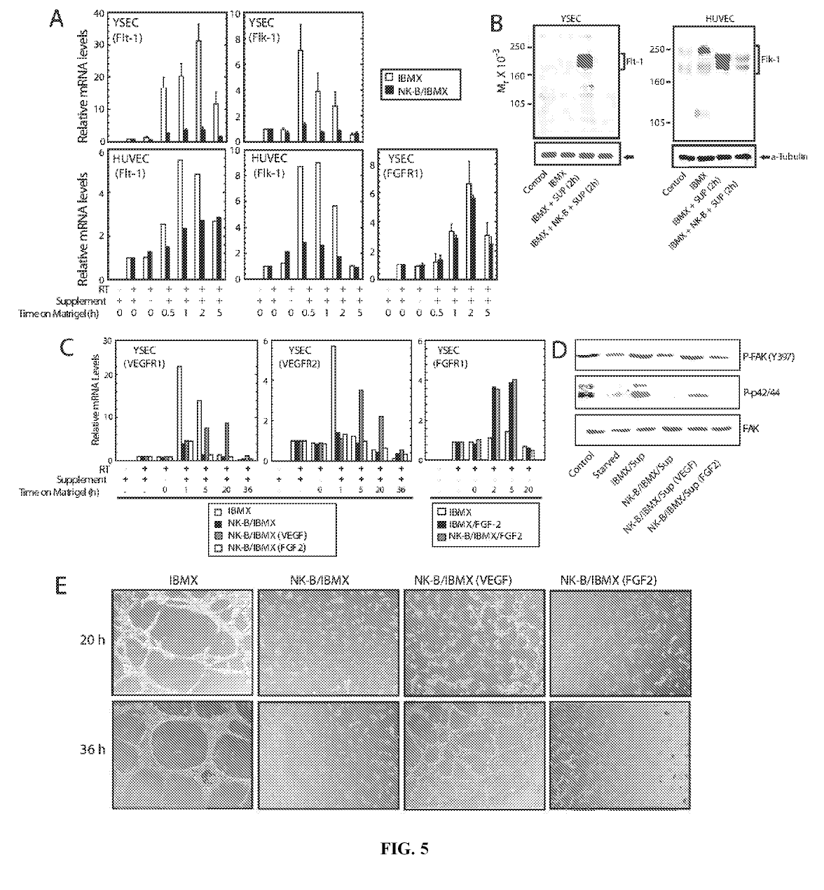

Vasoactive endothelial cell growth factor (VEGF) is a known for its effects on endothelial cell network proliferation. Therefore, to further investigate the effects of NK-B, experiments were designed to investigate the effect of NK-B on VEGF In these experiments, YSECs and HUVECs were treated with IBMX or NK-B/IBMX in supplement-free medium for 1 or 2 h, respectively and then plated on Matrigel containing supplemented medium for 0.5, 1, 2, and 5 h, FIG. 5A. RNA was isolated, and transcripts were quantified by real-time RT-PCR. The transcript level in untreated cells was designated 1 (2-3 independent experiments). RT, reverse transcriptase; Flt-1 and Flk-1, Type I and Type II high affinity VEGF receptors respectively. FIG. 5B shows a Western blot analysis of Flt-1 and Flk-1 in YSECs and HUVECs. FIG. 5C, VEGF, but not FGF2, rescues Flt-1 and Flk-1 expression and cell signaling; left and middle panels, RT-PCR analysis of Flt-1 and Flk-1 expression. Right panel, YSECs were treated with IBMX or NK-B/IBMX in supplement-free media for 1 h. Treated cells were plated on Matrigel, with or without 100 ng/ml FGF2, for 0.5, 1, 2, and 5 h. RNA was isolated, and transcripts were quantitated by real-time RT-PCR. FIG. 5D, Western blot analysis of unphosphorylated and phosphorylated FAK and p42/p44 MAPK in cells analyzed in panel C. FIG. 5E, YSECs were treated with NK-B/IBMX for 1 h in supplement-free medium. Cell were treated with recombinant VEGF164 (100 ng/ml) and mouse FGF2 (100 ng/ml), incubated for 15 min, and then plated on Matrigel containing LSGS, LSGS/VEGF164, LSGS/FGF2. Vascular network assembly was assayed at the indicated times. Thus, taken together these data indicate that NK-B mediates its effect on network assembly not just through NK receptors, but also by down regulating both the Type I and Type II VEGF receptors.

### Example 19

### NK-B Increases Synthesis of the Anti-Angiogenic Protein Calreticulin

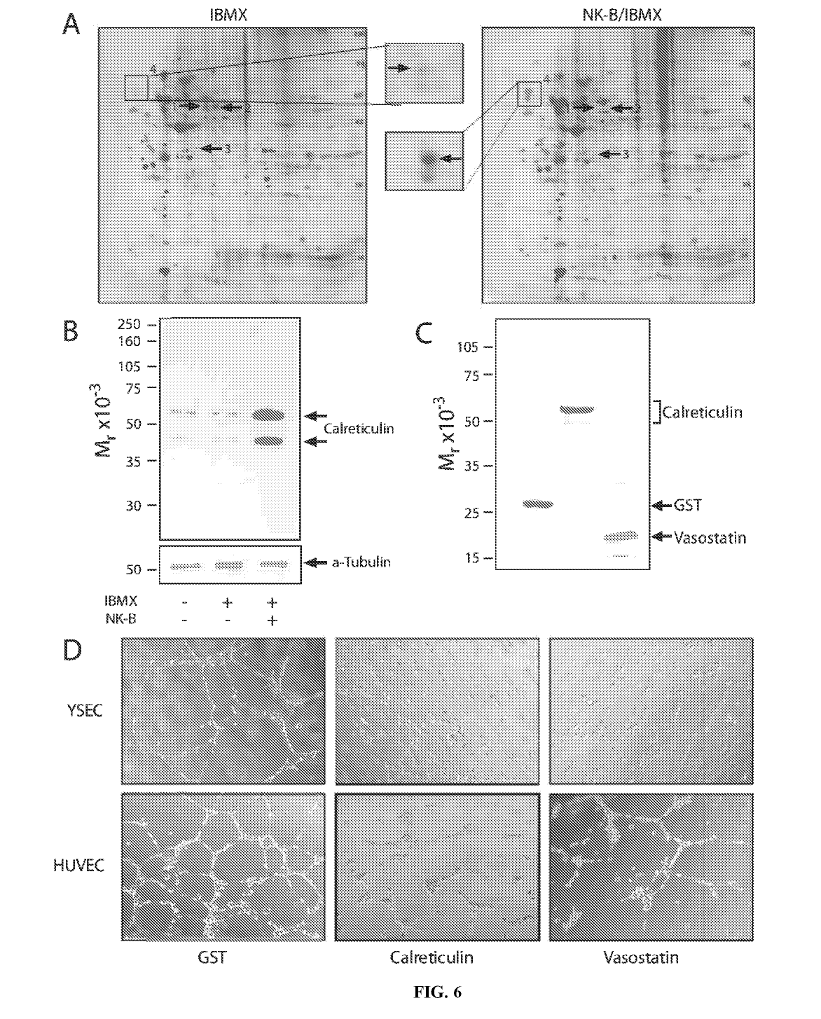

Because prior results show that the NK-B regulatory axis may work on many levels, the inventors also decided to investigate the effects of NK-B on the known anti-angiogenic protein calreticulin. In these experiments, YSECs were treated with IBMX or NK-B/IBMX for 1 h in supplement-free M200 medium and then 10 min in supplemented M200 medium, FIG. 6A. Whole cell extracts were subjected to 2D gel electrophoresis and stained with Coomassie blue. Representative stained gels are shown, and the inset shows the spot identified as calreticulin. FIG. 6B shows a Western blot analysis of calreticulin in whole cell lysates of YSECs after treating with vehicle, IBMX, or NK-B/IBMX for 1 h in supplement-free medium, followed by 10 min in supplemented medium. Blots were probed for calreticulin, and then stripped and reprobed for α-tubulin. As shown, treatment with NK-B/IBMX increased the expression of calreticulin. A representative blot of calreticulin and α-tubulin is shown. FIG. 6C, SDS-PAGE of E. coli overexpressed and purified recombinant calreticulin and vasostatin. FIG. 6D confirms that, in vitro, purified calreticulin and vasostatin inhibit YSEC and HUVEC vascular network assembly. Cells were treated with GST (10 μg/ml), calreticulin (13 μg/ml), or vasostatin (5 μg/ml) for 1 h in supplement-free M200 medium and plated on Matrigel to assess vascular network assembly. Thus, the data indicate that NK-B effect on angiogenesis is also mediated via its effect on the expression of calreticulin.

### Example 20

### NK-B and TXA-2 Signaling Synergistically Opposes Vascular Network Assembly

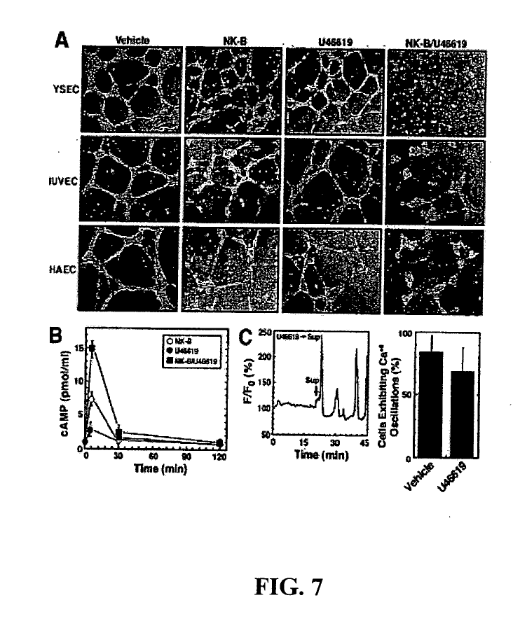

Furthering their investigations into agents involved in the NK-B axis, the inventors studied the effects of NK-B and thromboxane A2 (a vasoconstrictive agent) on vascular network assembly. In these investigations, YSECs, HUVECs, and HAECs were treated with vehicle, the thromboxane mimetic U46619 (2 μM) (Calbiochem, LaJolla, Calif.), or NK-B/U46619 in supplement-free medium for 1 or 2 h, respectively, plated on Matrigel containing supplemented medium, and incubated for 16 h at 37° C. to assess vascular network assembly, FIG. 7A. These experiments show that, while NK-B mildly disrupted network assembly and U46619 had little effect, when NK-B and U46619 were given together network assembly was almost completely abolished. FIG. 7B, YSECs were treated with NK-B, U46619, or NK-B/U46619 in supplement-free M200 medium, and cAMP was quantitated various times thereafter (mean+/−S.E., 3 independent experiments). FIG. 7C, real-time measurements of Ca⁺² oscillations. (Sup, supplemented M200 medium). The graphs depict the % of cells exhibiting oscillations (left) and the fluorescence intensity at time 0 (F₀) (right). Data illustrated in FIGS. 7 B and C show that NK-B and U46619 both decrease the amount of cAMP while decreasing the amount of Ca²⁺ oscillations.

### Example 21

### Anti-Angiogenic NK-B/TXA2 Regulatory Axis

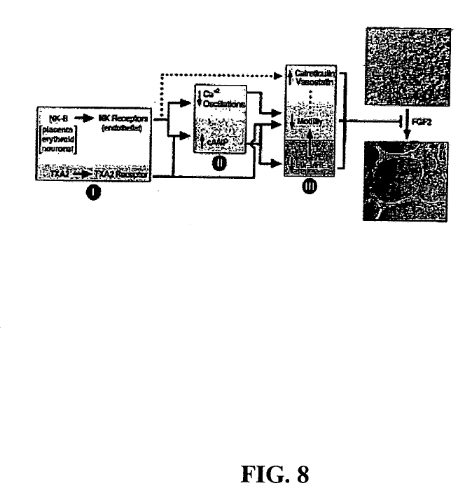

Based on the experimental data the inventors have developed a model of the anti-angiogenic NK-B/TXA2 regulatory axis. FIG. 8 shows extracellular (I), intracellular signaling (II), and effector (III) modules, each consisting of multiple reactions, which collectively oppose angiogenesis. NK-B, expressed in neurons, erythroid cells, and the placenta, signals through NK receptors to increase cAMP and to ablate Ca⁺² oscillations. The functional consequences of this signaling include increased calreticulin expression, decreased cell motility, and Flk1 and Flt-1 downregulation. Though it is unclear how many convergence points exists between NK-B and TXA2 signaling pathways, TXA2 signaling potentiates NK-B-mediated cAMP induction and the cell motility blockade and synergizes with NK-B to oppose vascular network assembly. The dotted line denotes a putative pathway that is not yet supported by experimental evidence. As both NK-B and TXA2 are upregulated in preeclampsia, the NK-B/TXA2 regulatory axis establishes a mechanistic foundation for understanding how these factors functionally interact via direct actions on vascular endothelium.

### Example 22

### NK-B Alone Decreases the Kinetics of Vascular Network Assembly

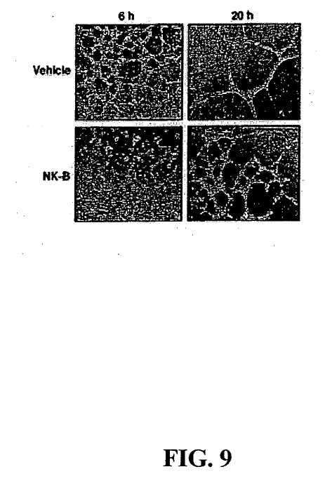

While the previously described investigations report the effect of NK-B in synergy with other active compounds, the inventors wanted to assess the effect of NK-B alone on vascular network assembly. Thus, YSECs were treated with vehicle (0.5% DMSO) or NK-B (100 μM) for 1 h in supplement-free M200 medium, plated on Matrigel containing supplemented medium, and incubated for 20 h at 37° C. FIG. 9 shows representative digital images captured at 6 and 20 h. These data show that, starting as early as 6 hours, cells treated with NK-B had greatly reduced network assembly while by 20 hours the vascular networks appeared more similar to the control at 6 hours.

### Example 23

### NK-B/IBMX Abrogates Vascular Network Assembly in Three-Dimensional Collagen Gels

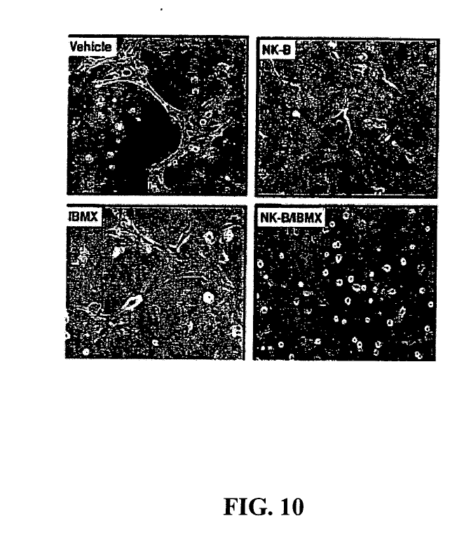

As a further investigation into the effects of NK-B, IBMX and NK-B/IBMX on vascular network assembly HUVECs were treated with vehicle, IBMX (100 μM), NK-B (100 μM), or NK-B/IBMX for 1 h in supplement-free M200 medium. Cells were then mixed with 500 μl of 2 mg/ml neutralized Collagen I (rat tail; BD Biosciences), transferred into 12-well plates, and allowed to solidify at 37° C. Supplemented medium containing the indicated reagents was added on top of the gel, cells were incubated for 4 days at 37° C., vascular network assembly was analyzed, and digital images were captured and are shown in FIG. 10.

### Example 24

### VEGF164-Dependent Angiogenesis Dominates Over Anti-Angiogenic Activity of NK-B/IBMX

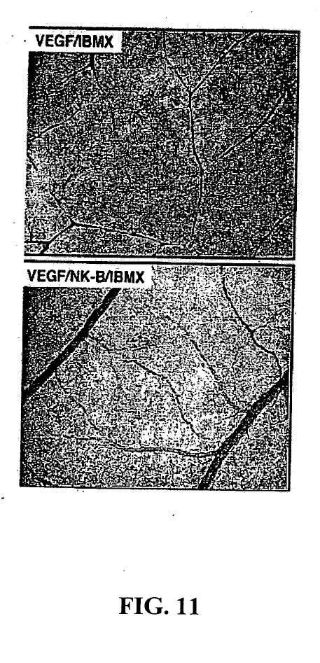

Further delineating the anti-angiogenic effects of NK-B the inventors designed experiments testing the effects of NK-B in combination with known angiogenic agents. Thus, the inventors tested the in-vivo effects of treating the chick embryo CAM with the known angiogenic agent VEGF in concert with NK-B/IBMX. FIG. 11 shows that in the CAM VEGF164-dependent angiogenesis (top panel) dominates over the anti-angiogenic activity of NK-B/IBMX (bottom panel). Methylcellulose containing VEGF164 (200 ng)/IBMX (100 μg) or VEGF164 (200 ng)/NK-B (40 μg)/IBMX was applied to the CAM of day 7 chicken embryos. After 48 h, blood vessels were analyzed, digital images were captured at 40× magnification, and representative images are shown (12-15 eggs were analyzed per condition, 3 independent experiments).

### Example 25

### Forskolin Partially Disrupts YSEC but not HUVEC Vascular Assembly

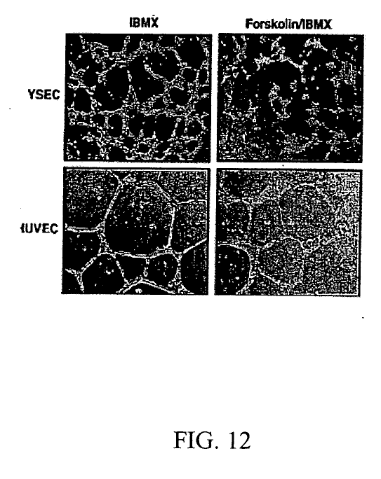

Continuing their investigations into the NK-B regulatory axis, the inventors designed experiments to study the effects of forskolin, a known agent for increasing cAMP on the disruption of vascular network assembly. In this experiment YSECs and HUVECs were treated with IBMX (100 μM) or forskolin (10 μM)/IBMX for 1 h in supplement-free M200 medium, plated on Matrigel containing supplemented medium, and incubated for 16 h and 12 h for YSECs and HUVECs, respectively at 37° C. Digital images were captured, and representative images are shown in FIG. 12. As shown, like NK-B, forskolin/IBMX disrupts network assembly in YSECs, however unlike NK-B forskolin/IBMX has no effect on HUVECs. These data indicate that the two agents may be acting through different mechanisms.

### Example 26

### Forskolin, but not Calreticulin and Vasostatin, Downregulates Flt-1 and Flk-1

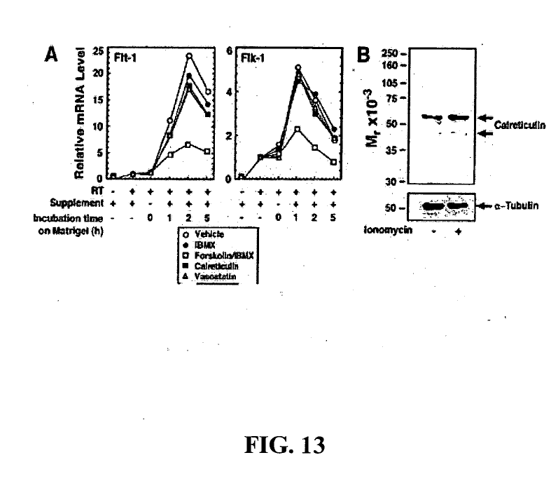

To determine the whether a perturbation in VEGF was responsible for the changes in vascular assembly, the inventors investigated the effect on transcription of Flt-1 and Flk-1, the major VEGF receptors. FIG. 13A, YSECs were treated with vehicle, IBMX (100 μM), forskolin (10 μM)/IBMX, calreticulin (13 μg/ml), vasostatin (5 μg/ml) in supplement-free M200 medium for 1 h. Cells were then plated on Matrigel containing supplemented medium and the indicated reagents for 1, 2, or 5 h. RNA was isolated, and quantitative RT-PCR analysis was conducted to measure Flt-1 and Flk-1 mRNA levels. RNA from untreated cells was used as a control, and the expression level in untreated cells was designated as 1 (mean, 2 independent experiments). RT, reverse transcriptase. FIG. 13B, Ca⁺² ionophore ionomycin weakly increases calreticulin synthesis. Endogenous calreticulin was measured by Western blotting in whole cell extracts from YSECs treated with vehicle (0.1% DMSO) or with 2 μM lonomycin for 1 h in supplement-free M200 medium. Western blots were stripped and reprobed to detect alpha-tubulin. The calreticulin/alpha-tubulin ratio increased ˜2-fold upon ionomycin treatment. These data illustrate that while forskolin decreases the vascular network assembly as shown in FIG. 12, its actions appear to be mediated by decreasing the transcription of the major VEGF receptors Flt-1 and Flk-1 while other compounds, including calreticulin failed to affect Flt-1 and Flk-1 transcription.

### Example 27

### NK-Inhibitor Induces Angiogenesis In Vivo

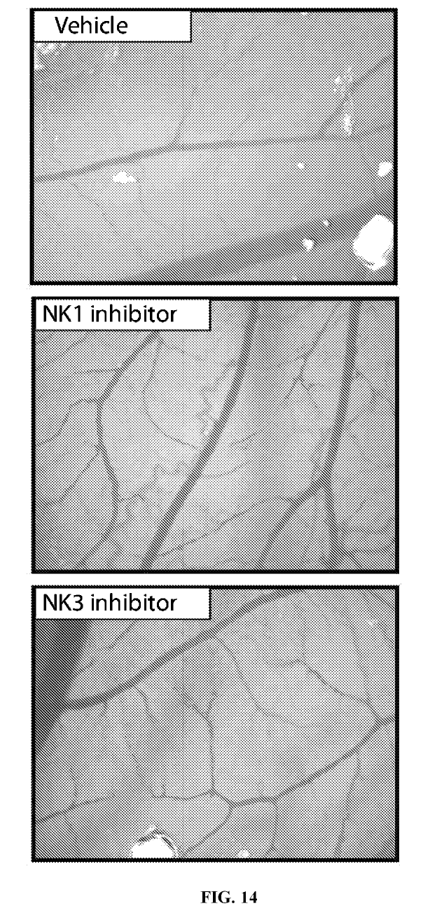

The preceding experiments illustrated that NK-B inhibitors could induce angiogenesis in vitro. Therefore, the inventors designed an experiment to test this effect in vivo. Thus, the inventors investigated whether NK-inhibitor induced angiogenesis in the chicken chorioallantoic membrane in vivo. Methylcellulose containing vehicle (FIG. 14, top) (0.1% DMSO) or NK1 antagonist, L733060, (5 μM) (FIG. 14, middle) (Tocris Bioscience, Ellisville, Mo.) or the NK3 antagonist SB222200, (2 μM) (Tocris, Bioscience) (FIG. 14, bottom) was applied to the CAM of day 7 chicken embryos. After 48 h, blood vessels were analyzed, digital pictures were captured at 40× magnification, and representative pictures are shown. Note that the microvessel density of CAM is much higher when treated with either the NK1 or the NK3 inhibitor. FIG. 14 demonstrates the ability of the NK1 and NK3 receptor antagonist to induce angiogenesis in the chick chorioallantoic membrane in vivo.

C. Inhibition of Angiogenesis

### Example 28

### NK-B Prevents Endothelial Cell Tube Formation

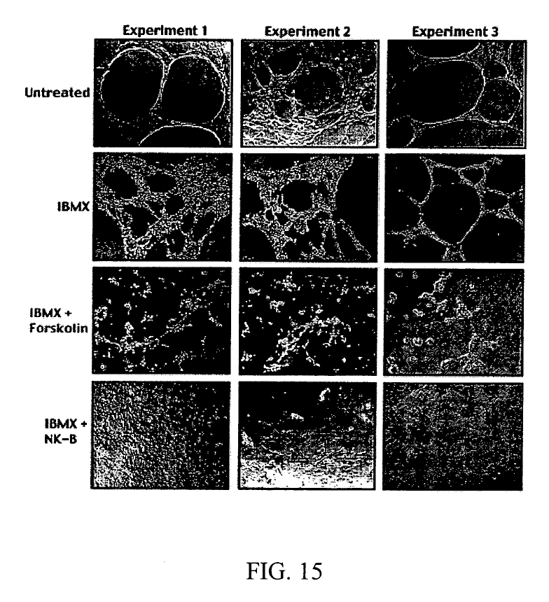

The preceding investigations prompt the question of the extent of the anti-angiogenic effects of NK-B. Referring to FIG. 15, YSEC cells treated either with solvent (0.5% DMSO in M200 medium), or 250 μM IBMX, or 250 μM IBMX+10 μM forskolin or with 250 μM IBMX+100 μM NK-B for 1 h in unsupplemented M200 medium and seeded on Matrigel in presence equal volume of LSGS containing M200 medium for capillary like tube formation and incubated for 20 h at 37° C. FIG. 15 shows results from 3 different experiments. These data show that while IBMX has little effect on vascular network assembly and IBMX/forskolin moderately disrupts vascular network assembly, NK-B/IBMX completely blocked vascular network assembly in each of the three experiments performed.

### Example 29

### NK-B Mediated Inhibition of Tube Formation is Reversible

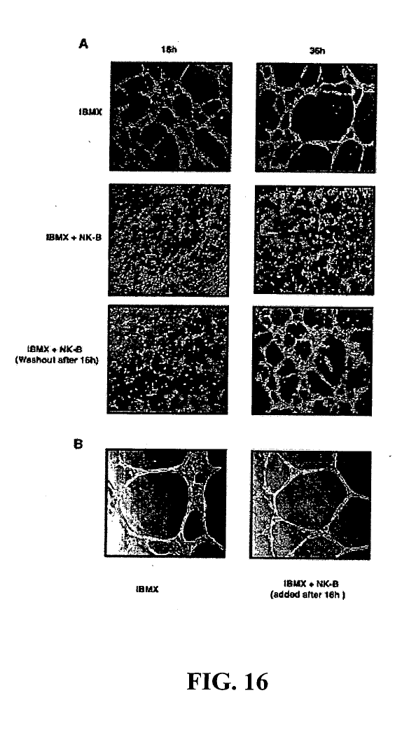

Further investigating the extent and mechanism of action of the NK-B effect on vascular network assembly the inventors studied the extent of NK-B inhibition. Referring to FIG. 16A, YSEC cells were treated with IBMX (250 μM) or IBMX (250 μM)+NKB (100 μM) 1 h in unsupplemented M200 medium and seeded on Matrigel in presence equal volume of LSGS containing M200 medium. Cells were incubated in presence of NK-B for 15 h and further incubated for 20 h in presence (middle panel) or absence of NK-B (bottom panel). Note that after removal of NK-B, YSEC cells started to form tube like structures indicating NK-B does not induce apoptosis and its effect is reversible. FIG. 16B, YSEC cells were treated with IBMX and seeded on Matrigel for 16 h for tube formation and further incubated for 20 h in presence or absence of NK-B. FIG. 16B shows that NK-B does not block the maintenance signal for already formed tubes. Thus, these investigations indicate that the ability to inhibit vascular network assembly is limited to current growth and does not disrupt or destroy prior networks.

### Example 30

### Cell Type Specific Effect of NK-B on Human Primary Endothelial Cells

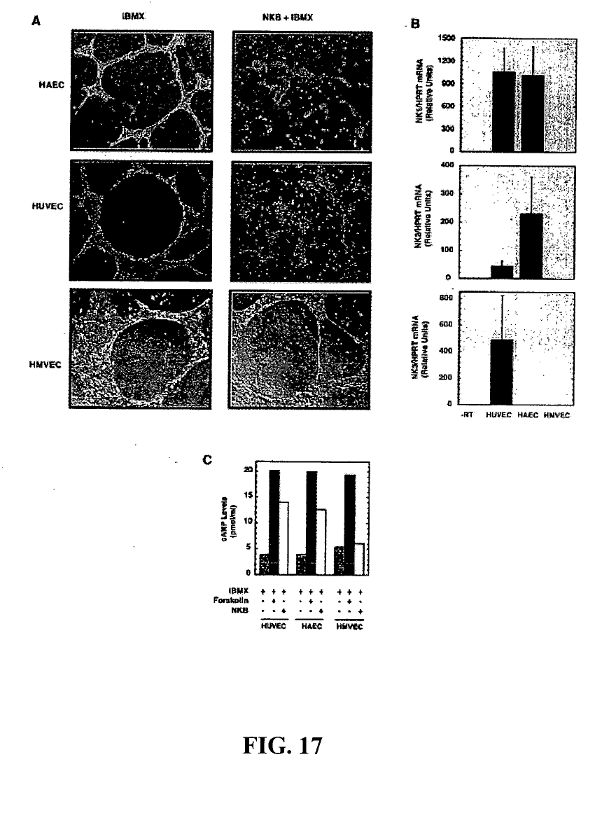

In order to investigate the specificity of the effect of NK-B on vascular assembly, several endothelial cell types were used. Referring to FIG. 17A, HAEC, HUVEC and HMVEC cells were treated with IBMX (250 μM) or IBMX+NK-B (100 μM) for 2 h in unsupplemented M200 medium and seeded on Matrigel in presence of LSGS containing M200 medium and incubated for 12 h. Note that NK-B had no effect on the tube formation ability on microvascular (HMVEC) cells. FIG. 17B depicts quantitative real time RT-PCR analysis of neurokinin receptor subtypes NK1, NK2, and NK3 levels in HUVEC cells, HAEC cells, and HMVEC cells. Relative levels of NK1, NK2 and NK3 mRNAs were normalized by the levels of HPRTtranscripts. The plots depict the mRNA/HPRT mRNA ratios (mean±S.E., three independent experiments) in which the ratios obtained from the RT receptors. FIG. 17 B shows that HMVECs which did not respond to NK-B do not display NK1, NK2 or NK3 receptors. FIG. 17C shows the results of HUVEC, HAEC, HMVEC cells treated with 250 μM IBMX with or without 10 μM forskolin or 100 μM NK-B for 1 h. cAMP levels were quantitated in cell lysates by a competitive immunoassay (mean±S.E., three independent experiments). These results indicate that the NK-B effect is cell specific and that the NK1, NK2 and NK3 receptors may be one of the prime receptors through which NK-B exerts its anti-angiogenic effects.

### Example 31

### NK-B Mediates its Function Through NK Receptors

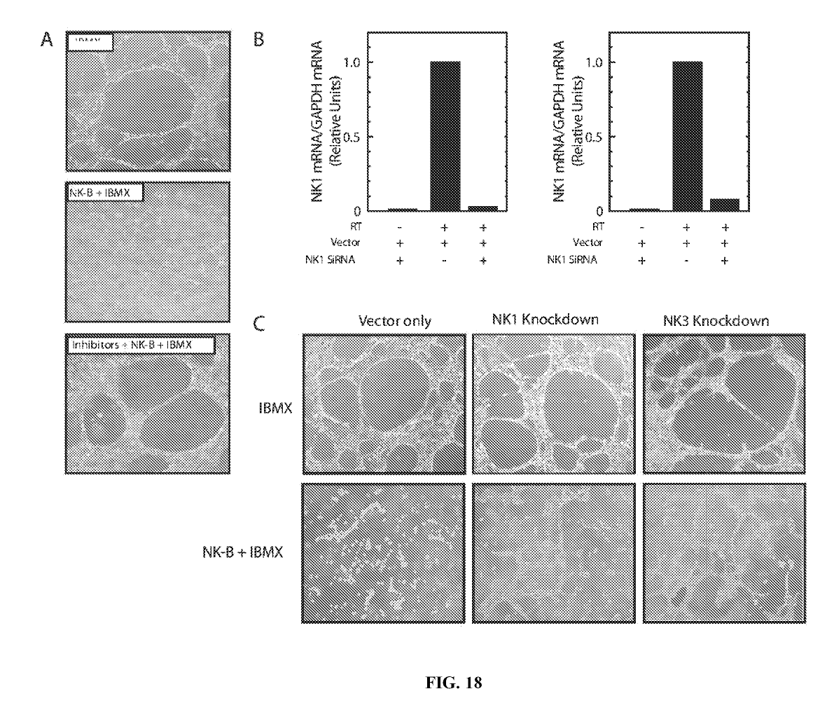

Building on the experiment described in Example 30, the inventors designed experiments to more clearly identify the mechanisms by which NK-B inhibits vascular network assembly. Referring to FIG. 18A, YSEC cells were plated on Matrigel for tube formation after treating them either with 250 μM IBMX (top), or with 250 μM IBMX+100 μM ML-B (middle) or with 2.5 μM NKa and 1 μM NK3 inhibitors for 15 minutes followed by 205 μM IBMX+100 μM NK-B (bottom). FIG. 18B shows quantitative RT-PCR analysis of NK1 (left) and NK3 (right) mRNA in stable clones of YSEC cells expressing NK1 and NK3 siRNA molecules (see Example 9). (RT means reverse transcriptase). FIG. 18C illustrates the results of the tube formation assay with stable clones of the YSEC cells analyzed in FIG. 18B. Note that knocking down of either NK1 or NK3 receptor partially blocks the NK-B effect.

### Example 32

### NK-B Prevents Endothelial Cell Migration

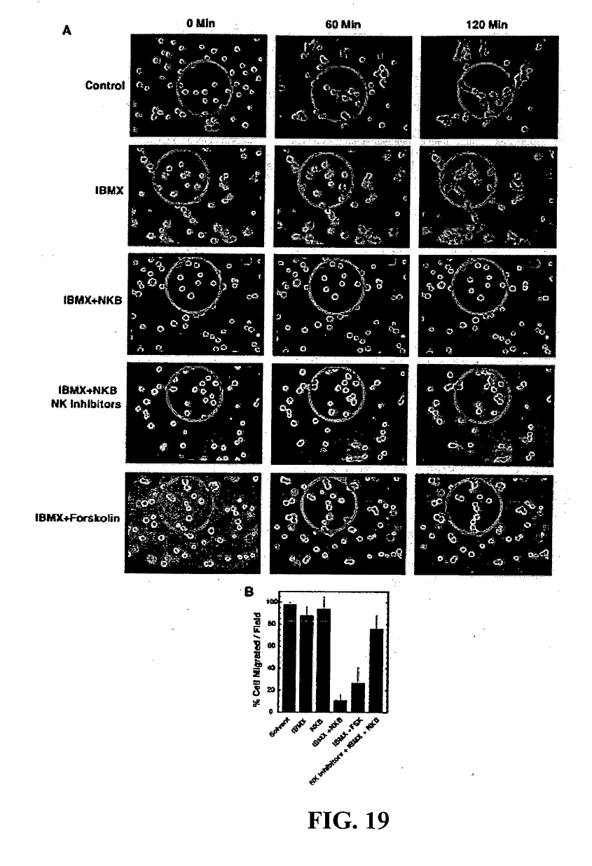

Referring to FIG. 19A, YSEC cells were treated with solvent, IBMX (μM), IBMX+NK-B (100 μM), IBMX+NK-B+NK1 inhibitor (2.5 μM)+NK3 inhibitor (1 μM) and IBMX+forskolin for 1 h and plated on Matrigel. Cells were allowed to adhere for 30 minutes and then cell migration was monitored using time lapse video micrograph for 2 h. FIG. 19A shows snapshots of representative frames of each condition at different time intervals. FIG. 19B is a histogram showing the resulting of the quantification of cells migrating in the condition illustrated in FIG. 19A. The data illustrated in FIG. 19B represents migrating YSEC cells in a single frame counted at different experimental conditions and calculated as a percent of total number cells in that frame. The plot shows results of four different experiments (mean±S.D.). This data indicates that NK-B exerts its effect, not by promoting apoptosis or by decreasing cell viability but, rather, by decreasing cell motility and adhesion.

### Example 33

### NK-B Mediated Induction of Calreticulin in Endothelial Cells

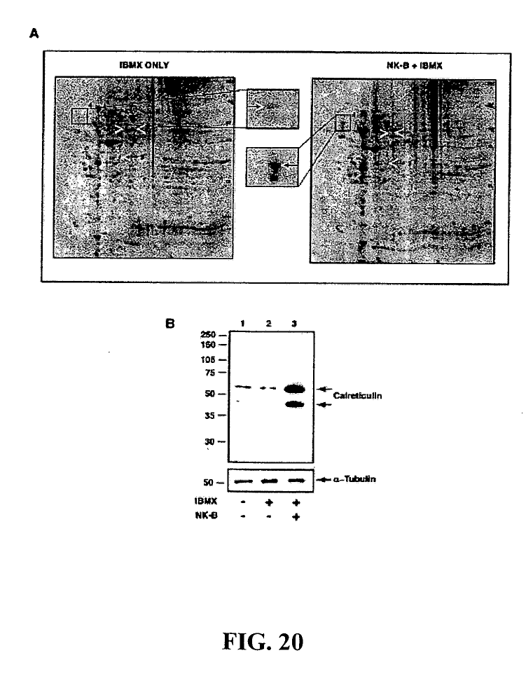

The inventors then preformed an experiment similar to that shown in FIG. 6 to further investigate the role by which NK-B mediates its effects. FIG. 20A shows YSEC cells that were treated either with IBMX or IBMX+NK-B for one 1 h in media without serum and growth factors and then incubated for 10 minutes in presence of low serum growth supplement (LSGS). Whole cell extracts were subjected to 2D gel electrophoresis and stained. FIG. 20A shows pictures of the stained gels. Inset shows the calreticulin band (identified by maldi mass spectrometry). Note that in presence of NK-B the intensity of the band increased. FIG. 20B depicts Western blot analysis of calreticulin in whole cell extracts of YSEC cells after treating with Vehicle (lane-1), IBMX alone (lane 2) and IBMX+NK-B (lane 3) for 1 h in unsupplemented media and 10 minutes in media containing LSGS. Blots were probed for calreticulin, stripped and probed for α-tubulin. One representative blot of Calreticulin and α-tubulin is shown. Note that the calreticulin antibody detected a 55 kd and a 40 kd band for full length and truncated calreticulin respectively. These results further confirm that NK-B exerts its anti-angiogenic effects through multiple pathways.

### Example 34

### NK-B Inhibits VEGF Receptor Transcription and Down Stream Signaling

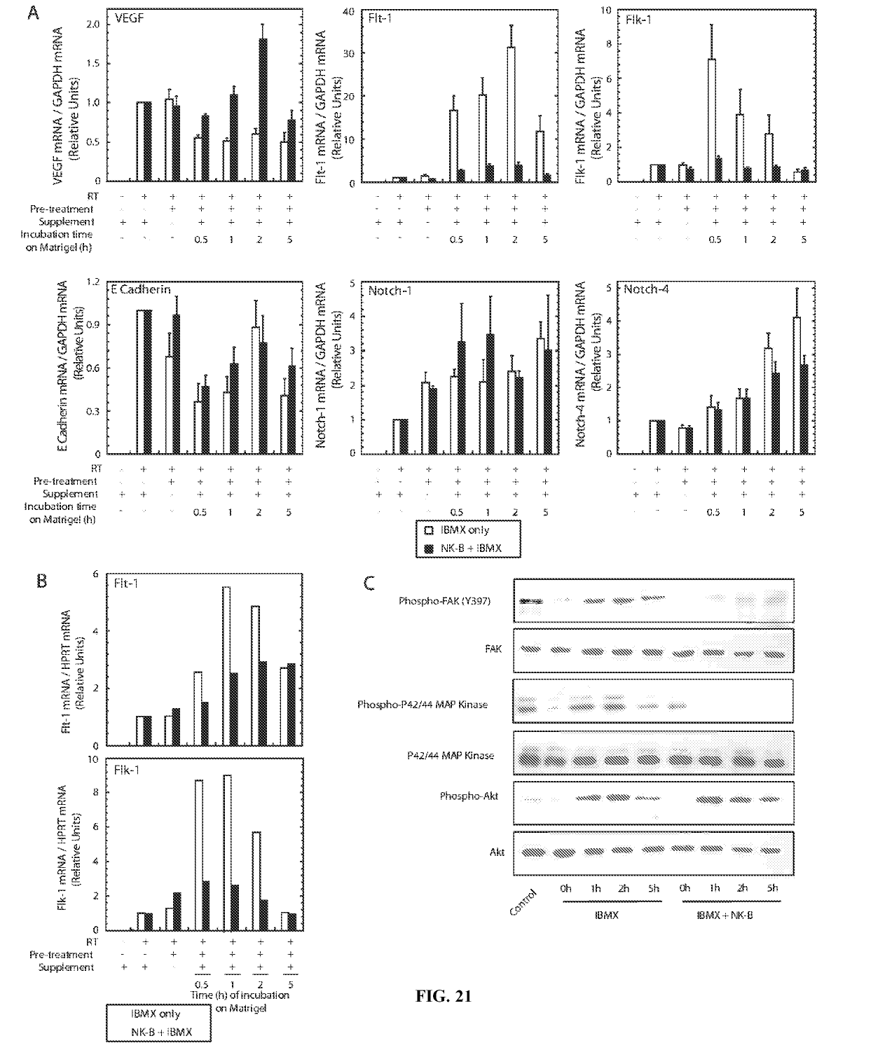

Referring to FIG. 21A, YSEC cells were treated with IBMX or IBMX+NK-B in unsupplemented media for 1 h. Treated cells were then plated on Matrigel in presence of supplemented media for 0.5 h, 1 h, 2 h, and 5 h. RNA was isolated and Quantitative RT-PCR analysis was done for different genes. RNA from untreated YSEC cells were used as a control and plots were generated considering the expression level in untreated cells as 1 (mean±S.E., three different experiments (RT is reverse transcriptase). Note that treatment with NK-B blocks transcriptional induction of both VEGF receptors Flt-1 and Flk-1. VEGF mRNA level increased upon NK-B treatment with no significant changes in E Cadherin, Notch-1 and Notch-4 transcription. FIG. 21B shows that HUVEC cells were treated with IBMX or IBMX+NK-B in unsupplemented media for 1 h. Treated cells were then plated on Matrigel in presence of supplemented media for 0.5 h, 1 h, 2 h, and 5 h. RNA was isolated and Quantitative RT-PCR analysis was done for Flk-1 and Flt-1 (mean, two different experiments). RNA from untreated YSEC cells were used as control and plots were generated considering the expression level in untreated cells as 1. FIG. 21 C illustrates Western blot analysis of the same cells analyzed in FIG. 21A. Samples were probed for both phosphorylated and nonphosphorylated forms of FAK kinase, p42/44 MAP kinase (ERK1/ERK2) and AKT. Note that NK-B treatment inhibits both FAK and ERK1/2 phosphorylation but not AKT phosphorylation.

### Example 35

### VEGF but not bFGF Rescues the NK-B Effect on Endothelial Cells

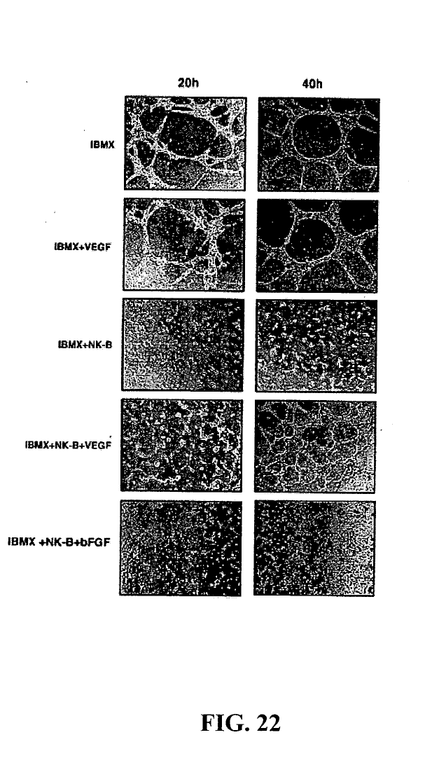

Referring to FIG. 22, YSEC cells were treated with IBMX+NK-B for 1 h in unsupplemented media. Cells were divided into three parts. In two parts 100 ng/ml mouse VEGF164 and 30 ng/ml mouse bFGF were added respectively. Cells were further incubated for 15 minutes and then plated on Matrigel in presence of LSGS+(100 ng/ml) VEGF164 or LSGF or just LSGS and monitored for tube formation at different time intervals. Note that in presence of both VEGF and NK-B cells can form capillary-like tubes but in a slower kinetics.

### Example 36

### NK-B Suppresses Endothelial Cell Motility and Vascular Network Assembly

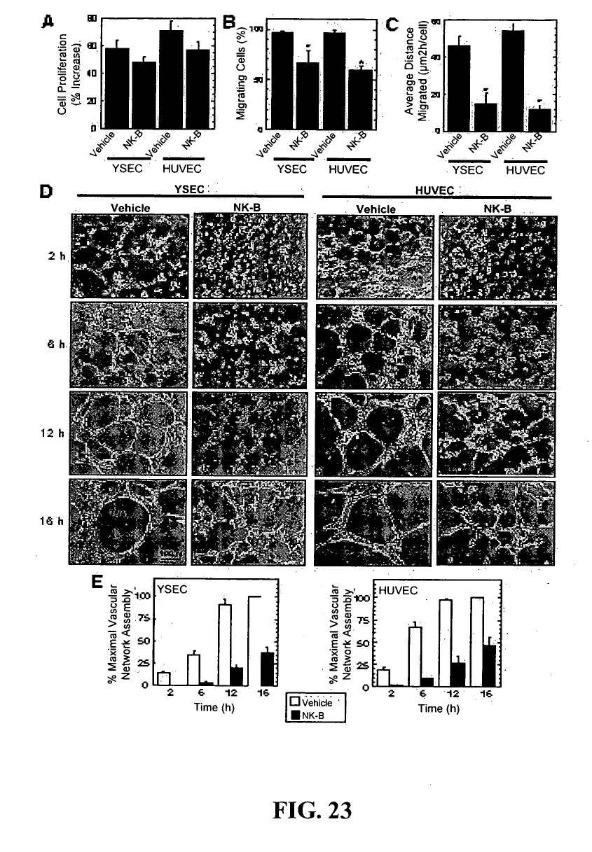

YSECs and HUVECs were plated in supplement-free medium for 24 h and treated with vehicle, or NK-B for 1 h. Cells were incubated with supplemented medium containing vehicle or NK-B for 24 h, and proliferation was quantitated. FIG. 23A depicts the percent increase of proliferation relative to cells grown without supplement (mean+/−S.E., 3 independent experiments). YSECs and HUVECs were treated with vehicle or NK-B in supplement-free medium for 1 and 2 h respectively and plated on Matrigel with supplemented medium containing the same reagents. Cell migration was monitored for 2 h. Migrating cells were expressed as a percent of the total cells (mean+/−S.E., 4 independent experiments) as shown in FIG. 23B. (p<0.05). The rate of migrating YSECs and HUVECs analyzed in FIG. 23B was measured using Slidebook 4 software. YSECs and HUVECs were treated with vehicle or NK-B for 1 and 2 h respectively in supplement-free medium, plated on Matrigel containing supplemented medium, and incubated for 16 h at 37° C. Representative pictures are shown in FIG. 23D. The length of tubular structures from three adjacent frames was quantitated from 3 independent experiments. The length of the structures in vehicle-treated cells at 16 h was designated 100% as is shown in FIG. 23E. These data indicate that NK-B has little effect on cell proliferation. However there is a significant effect of NK-B alone on the migration of endothelial cells. Thus, while the effect of NK-B is not lethal to endothelial cells their ability to form vascular networks is severely inhibited.

### Example 37

### IBMX and TXA2 Potentiate NK-B Activity to Regulate Endothelial Cell Function

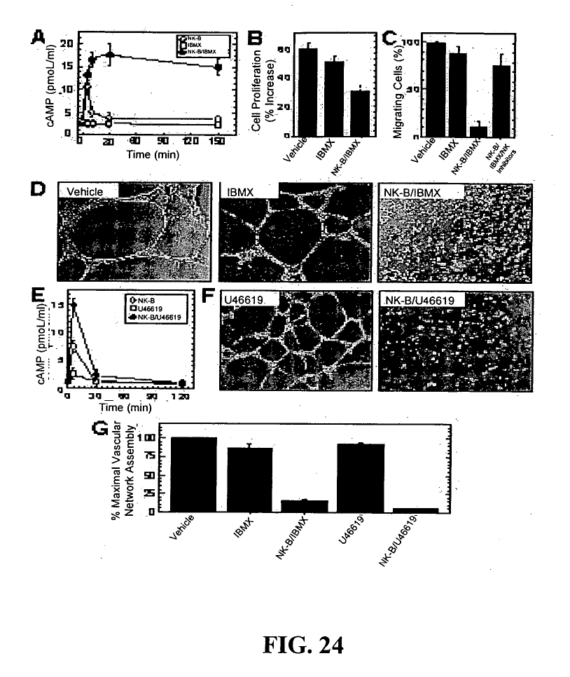

YSECs were treated with NK-B or NK-B/IBMX in supplement-free medium, and cAMP was assayed. FIG. 24A shows the data as mean+/−S.E., 3 independent experiments. YSECs were plated in supplement-free medium for 24 h and treated with vehicle, IBMX, or NK-B/IBMX for 1 h. Cells were incubated with supplemented medium containing the same reagents for 24 h, and proliferation was quantitated. FIG. 24B depicts the percent increase of proliferation relative to cells grown in supplement-free medium (mean+/−S.E., 3 independent experiments, p<0.05). YSECs were treated with the indicated reagents in supplement-free medium for 1 h and plated on Matrigel with supplemented medium containing the same reagents, and motility was monitored for 2 h. Migrating cells were expressed as a percent of total cells as shown in FIG. 24C (mean+/−S.E., 3 independent experiments). YSECs were treated with vehicle, IBMX, or NK-B/IBMX for 1 h in supplement-free medium, plated on Matrigel containing supplemented medium, and incubated for 20 h at 37° C. Representative micrographs are shown in FIG. 24D. YSECs were treated with NK-B, U46619 (a thromboxane mimetic), or NK-B/U46619 in supplement-free medium. cAMP was quantitated various times thereafter FIG. 24E (mean+/−S.E., 3 independent experiments). YSECs were treated with U46619 or NK-B/U46619 in supplement-free medium for 1 h, and vascular network assembly on Matrigel. Representative micrographs are shown in FIG. 24F. Quantitation of vascular network assembly of cells was then made as shown in FIG. 24E. This data indicates that thromboxane also potentiates the effects of NK-B.

### Example 38

### NK-B Inhibits Vascular Remodeling of Certain Human Endothelial Cell Subtypes

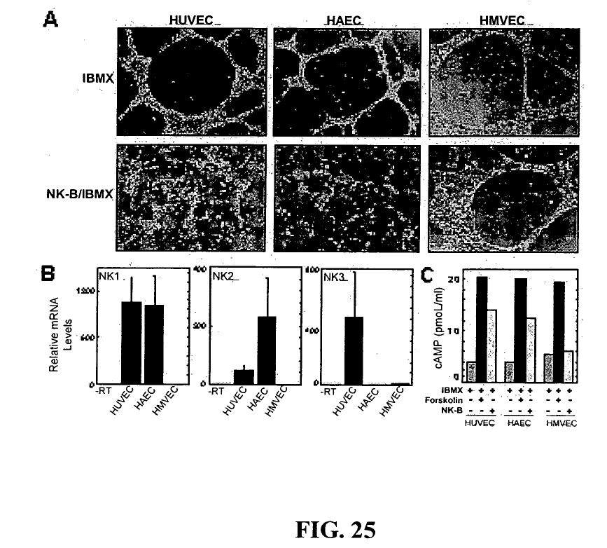

HUVEC, HAEC, and HMVEC cells were treated with IBMX or NK-B/IBMX for 2 h in supplement-free medium, plated on Matrigel containing supplemented medium, and incubated for 12 h. Representative micrographs are shown in FIG. 25A. Quantitative real-time RT-PCR analysis of NK1, NK2, and NK3 receptor mRNA levels in HUVECs, HAECs, and HMVECs were performed to determine the expression of each receptor. The relative mRNA levels were normalized by HPRT mRNA levels. FIG. 25B depict the mRNA ratios in which the ratios obtained for the −RT condition were designated as 1 (mean+/−S.E., 3 independent experiments). HUVECs, HAECs, HMVECs were treated with IBMX with or without forskolin or NK-B for 1 h. cAMP was quantitated in cell lysates and this data is shown in FIG. 25C (mean+/−S.E., 3 independent experiments). This data indicates that NK receptors are one mechanism through which NK-B exerts it effects.

### Example 40

### NK-B is Anti-Angiogenic and Disruption of Endogenous Neurokinin Signaling is Angiogenic in the Chicken Chorioallantoic Membrane

Methylcellulose containing vehicle, FGF2 (500 ng)/IBMX (100 μg), FGF2/NK-B (40 μg) with or without IBMX, FGF2/mNK-B/IBMX (mNK-B, inactive mutant of NK-B), or a combination of NK1-(L733060, 5 μM) and NK3-(SB222200, 2 μM) selective inhibitors was applied to the CAM of day 7 chicken embryos. After 48 h, digital images were captured at 40× magnification, and representative images are shown in FIG. 2A (15-20 eggs were analyzed per condition, mean+/−S.E., 3-4 independent experiments). FIG. 2B depicts the relative microcirculation density (p<0.05). These data further confirm that the presence of the NK receptors is necessary for NK-B to exert its effects

### Example 41

### NK Receptor Requirement for NK-B-Mediated Abrogation of Vascular Network Assembly

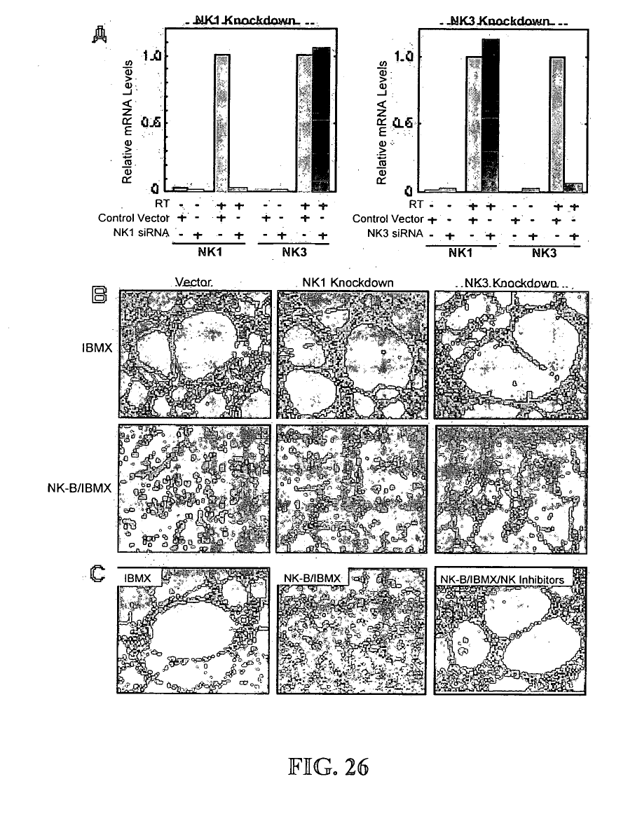

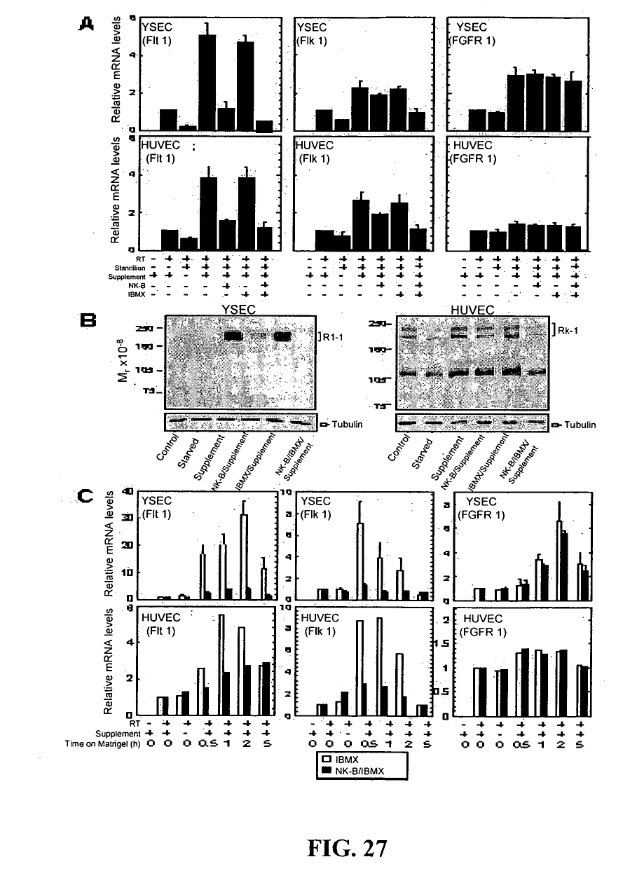

FIG. 26A shows the results of quantitative RT-PCR analysis of NK1 and NK3 mRNA in YSEC clonal lines stably expressing NK1 (left panel) and NK3 (right panel) siRNA molecules. (RT, reverse transcriptase). (B) Control and siRNA-expressing YSECs were treated with IBMX or NK-B/IBMX and incubated on Matrigel for 20 h at 37° C. Knocking-down either NK1 or NK3 reduced the capacity of NK-B/IBMX to abrogate vascular network assembly as illustrated by the representative micrographs shown in FIG. 27B. YSECs were treated with IBMX or NK-B/IBMX with or without NK1-(L733060) and NK3-(SB222200) selective inhibitors for 15 min, followed by NK-B/IBMX, and plated on Matrigel to assess vascular network assembly as shown in FIG. 27C. These results show that IBMX alone does not inhibit vascular assembly while NK-B does. The data further illustrate that the NK1 and NK3 receptors modulate NK-B activity as shown in FIG. 26B, the effect of NK1 or NK3 knockdown is an intermediate disruption of vascular network assembly. FIG. 26C shows that when both receptors are blocked, there is no difference compared to control because addition of specific inhibitors L733060 and SB222200 results in inhibition of the NK-B effect.

### Example 42

### NK-B Downregulates VEGF Receptors

To investigate the effect of NK-B on VEGF receptors, subconfluent YSEC and HUVEC cells were starved of supplement overnight and treated with the reagents as shown in FIG. 27A in supplement-free medium for 1 and 2 h respectively. Cells were then treated with supplemented medium containing the indicated reagents for 6 h. Transcripts were quantitated by real-time RT-PCR. The transcript level in untreated cells was designated 1 (2-3 independent experiments). RT, reverse transcriptase. FIG. 27B shows the results of a Western blot analysis of Flt-1 and Flk-1 in YSECs and HUVECs respectively quantified in FIG. 27A. YSECs and HUVECs were treated with IBMX or NK-B/IBMX in supplement-free medium for 1 and 2 h respectively and then plated on Matrigel containing supplemented medium for 0.5, 1, 2, and 5 h. Transcripts were quantitated by real-time RT-PCR as shown in FIG. 27C. The transcript level in untreated cells was designated 1 (2-3 independent experiments). RT, reverse transcriptase. As previously described, the inventors have found that NK-B mediates its effects on angiogenesis by different pathways. In this example, the inventors have found that NK-B down regulates the major receptors, Flk-1 and Flt-1 of the known angiogenic agent VEGF.

### Example 43

### NK-B Induces the Anti-Angiogenic Protein Calreticulin

To test the effects of NK-B on the known anti-angiogenic protein calreticulin, YSECs were treated with IBMX or NK-B/IBMX for 1 h in supplement-free medium and then 10 min in supplemented medium. Whole cell extracts were subjected to 2D gel electrophoresis and stained with Coomassie blue. Representative stained gels are shown in FIG. 6A, and the inset shows the spot identified as calreticulin. The cells incubated with NK-B illustrate much higher induction of calreticulin than do those cells incubated in IBMX alone. To confirm the induction of calreticulin, Western blot analysis of the lysate was performed. Representative Western blots of calreticulin and α-tubulin in whole cell lysates of YSECs after treatment with vehicle, IBMX, or NK-B/IBMX for 1 h in supplement-free medium, followed by 10 min in supplemented medium are shown in FIG. 6B. SDS-PAGE of purified recombinant calreticulin and vasostatin is shown in FIG. 6C. YSEC and HUVEC cells were treated with GST (10 μg/ml), calreticulin (13 μg/ml), or vasostatin (5 μg/ml) for 1 h in supplement-free M200 medium and plated on Matrigel to assess vascular network assembly. FIG. 6D are representative micrographs showing that calreticulin and vasostatin inhibit vascular network assembly.

As described herein, the inventors have identified and characterized an NK-B regulatory axis which has potent effects on angiogenesis. Modulations of the NK-B regulatory axis results in an increase in angiogenesis or a decrease in angiogenesis. Further, the inventors has shown that the NK-B regulatory axis is effective in various endothelial cell cultures as well as in vivo in the chick embryo, a model that is recognized by those of skill in the art as an advantageous model for studying the effects of angiogenesis. See, for example, Villamor et al., Bio. Neonate (2005)88:156-163, incorporated by reference herein in its entirety for all purposes. In addition, those of skill in the art have recognized a wide variety of disease which result from aberrant angiogenesis. Such disease may be the result of increased angiogenesis such as, for example, in tumorigenesis and macular degeneration. In addition, diseases of aberrant angiogenesis may result in decreased angiogenesis, such as, for example, as is the case with preeclampsia and peripheral vascular disease.

Therefore, the present disclosure has described compositions and methods to modulate angiogenesis in disease states where a decrease in angiogenesis is desirable or where an increase in angiogenesis is desirable. In addition, while the invention disclosed herein is useful in treating patients in need of modulating angiogenesis, the present invention is also useful as a tool in further delineating the NK-B regulatory axis, the molecular dynamics of angiogenesis and cell types and receptors that may be used to modulate angiogenesis. While this invention has been described in conjunction with the various exemplary embodiments outlined above, various alternatives, modifications, variations, improvements and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments.

What is claimed is:

1. A method for modulating the angiogenic activity of cells comprising contacting cells capable of angiogenesis with an effective amount of NK-B, an NK-B analog, an NK receptor agonist or an NK receptor antagonist wherein the angiogenic activity of the cells is increased or decreased.

2. The method of claim 1, wherein the method utilizes an NK receptor antagonist resulting in an increase in angiogenic activity.

3. The method of claim 2, wherein the NK receptor antagonist is selected from the group consisting of L733060, CP99994, MK869, SDZ NKT 343, GR 82334, L-732,138, RP 67580, Spantide I, WIN51708, SR142801, SB235375, SB218795, SB222200, MNK-B and combinations thereof.

4. The method of claim 1, wherein the method utilizes NK-B (SEQ ID NO: 1) or an NK-B agonist resulting in a decrease in angiogenic activity.

5. The method of claim 1 wherein the method utilizes an NK receptor agonist resulting in a decrease in angiogenic activity.

6. The method of claim 1, wherein the method is carried out on cells in an in vitro setting.

7. The method of claim 1, wherein the method is carried out on cells in an in vivo setting.

8. The method of claim 5, wherein the NK receptor agonist is selected from the group consisting of cycloseptide, C14TKL-1, GR 73632, hemokinin and [Sar⁹-Met(O₂)¹¹]-Substance P, senktide, [MePhe7] neurokinin B and combinations thereof.

9. A method of modulating angiogenesis in a patient in need thereof, comprising administering a pharmaceutical composition having (a) an effective amount of NK-B, an NK-B analog, an NK receptor agonist or an NK receptor antagonist and (b) a pharmaceutically acceptable carrier.

10. The method of claim 9, wherein the method utilizes an NK receptor antagonist resulting in an increase in angiogenesis in said patient.

11. The method of claim 10, wherein the NK receptor antagonist is selected from the group consisting of L733060, CP99994, MK869, SDZ NKT 343, GR 82334, L-732,138, RP 67580, Spantide I, WIN51708, SR142801, SB235375, SB218795, SB222200, mNK-B and combinations thereof.

12. The method of claim 9, wherein the method utilizes NK-B (SEQ ID NO: 1) or an NK-B agonist resulting in a decrease in angiogenesis in said patient.

13. The method of claim 12, wherein the NK-B agonist is selected from the group consisting of cycloseptide, C14TKL-1, GR 73632, hemokinin and [Sar⁹-Met(O₂)¹¹]-Substance P, senktide, [MePhe7] neurokinin B and combinations thereof.

14. The method of claim 9, wherein the pharmaceutical composition is administered orally, rectally, subcutaneously, parenterally, transdermally, topically or via a timed release implant.

15. The method of claim 9, wherein the patient in need suffers from rheumatoid arthritis, diabetic retinopathy, macular degeneration, atherosclerosis, psoriasis, tumor growth/metastasis, coronary artery disease, peripheral vascular disease, varicose veins or preeclampsia.

16. A composition for use in modulating angiogenesis comprising: (a) an effective amount of an angiogenesis modulating agent selected from the group consisting of NK-B (SEQ ID NO: 1), an NK-B analog, an NK receptor agonist, an NK receptor antagonist and combinations thereof; and (b) a pharmaceutically acceptable carrier.

17. The composition of claim 16, wherein the angiogenesis modulating agent is SEQ ID NO: 1, cycloseptide, C14TKL-1, GR 73632, hemokinin, [Sar⁹-Met(O₂)¹¹]-Substance P, senktide, [MePhe7] neurokinin B, L733060, CP99994, MK869, SDZ NKT 343, GR 82334, L-732,138, RP 67580, Spantide I, WIN51708, SR142801, SB235375, SB218795, SB222200 or combinations thereof.

18. A method of inhibiting blood vessel morphogenesis comprising contacting cells capable of blood vessel morphogenesis with a blood vessel morphogenesis-inhibitory amount of an isolated neurokinin-B peptide (SEQ ID NO: 1), analogs thereof or an NK receptor agonist.

19. The method according to claim 18, wherein the cells capable of blood vessel morphogenesis are endothelial cells.

20. The method according to claim 18, wherein the method is carried out in an in vivo setting.

21. The method according to claim 18, wherein the method is carried out in an in vitro setting.

22. The method according to claim 18, wherein the cells capable of blood vessel morphogenesis promote or maintain a pathologic state upon forming blood vessels.

23. The method of claim 22, wherein the pathologic state is cancer, rheumatoid arthritis, hemangioma, psoriasis, or ocular disease.

24. The use of NK-B (SEQ ID NO: 1), an NK-B analog, an NK receptor agonist, or an NK receptor antagonist in the manufacture of a medicament for the treatment of an angiogenesis related condition.

25. The use of claim 24, wherein the medicament is used in treating rheumatoid arthritis, diabetic retinopathy, macular degeneration, atherosclerosis, psoriasis, tumor growth/metastasis, coronary artery disease, peripheral vascular disease, varicose veins or preeclampsia.

26. The use of claim 24, wherein the NK receptor agonist or antagonist is, cycloseptide, C14TKL-1, GR 73632, hemokinin and [Sar⁹-Met(O₂)¹¹]-Substance P, senktide, [MePhe7] neurokinin B, L733060, CP99994, MK869, SDZ NKT 343, GR 82334, L-732,138, RP 67580, Spantide I, WIN51708, SR142801, SB235375, SB218795, SB222200 or combinations thereof.


SEQUENCE LISTING (29 sequences)

SEQ ID NO: 1 - protein, 10 aa, Homo sapiens
DMHDFFVGLM

SEQ ID NO: 2 - DNA, 19 bp, ARTIFICIAL
  siRNA for mouse NK1 cDNA.
caacaggacttacgagaaa

SEQ ID NO: 3 - DNA, 19 bp, ARTIFICIAL
  siRNA 1044-1061 mouse NK3 cDNA.
agatttcgtgcaggcttca

SEQ ID NO: 4 - DNA, 19 bp, ARTIFICIAL
  PCR primer
tgcccccatgtttgtgatg

SEQ ID NO: 5 - DNA, 19 bp, ARTIFICIAL
  PCR primer
ccttcccgagtactggtgt

SEQ ID NO: 6 - DNA, 20 bp, ARTIFICIAL
  PCR primer
ttgtcatctgggtcctggct

SEQ ID NO: 7 - DNA, 26 bp, ARTIFICIAL
  PCR primer
tgtagacacactgacacgactagatg

SEQ ID NO: 8 - DNA, 21 bp, ARTIFICIAL
  PCR primer
ccagaaggtcccaagcaacat

SEQ ID NO: 9 - DNA, 22 bp, ARTIFICIAL
  PCR primer
gtaggaccacatgacaaagggt

SEQ ID NO: 10 - DNA, 20 bp, ARTIFICIAL
  PCR PRIMER
tgaaggaagatggacggctg

SEQ ID NO: 11 - DNA, 23 bp, ARTIFICIAL
  PCR primer
cacaaagaagaaattgctgacct

SEQ ID NO: 12 - DNA, 19 bp, ARTIFICIAL
  PCR primer
tggactgagaccaagccca

SEQ ID NO: 13 - DNA, 20 bp, ARTIFICIAL
  PCR primer
tcattttcgttcctccgccc

SEQ ID NO: 14 - DNA, 22 bp, ARTIFICIAL
  PCR primer
ttggcaaatacaacccttcaga

SEQ ID NO: 15 - DNA, 21 bp, ARTIFICIAL
  PCR primer
gtcgcactaagactcctttcc

SEQ ID NO: 16 - DNA, 20 bp, ARTIFICIAL
  PCR primer
ttggaggctacaaggttcgc

SEQ ID NO: 17 - DNA, 21 bp, ARTIFICIAL
  PCR primer
cctaagacaccacggaagact

SEQ ID NO: 18 - DNA, 26 bp, ARTIFICIAL
  PCR primer
attggtggagatgatctctcaacttt

SEQ ID NO: 19 - DNA, 26 bp, ARTIFICIAL
  PCR primer
aacaccttctatattaactgtgaccg

SEQ ID NO: 20 - DNA, 21 bp, ARTIFICIAL
  PCR primer
agtcgtgtgcatgatcgaatg

SEQ ID NO: 21 - DNA, 20 bp, ARTIFICIAL
  PCR primer
acgaccactaaccgatacgt

SEQ ID NO: 22 - DNA, 21 bp, ARTIFICIAL
  PCR primer
tactccaccgtcaccatggac

SEQ ID NO: 23 - DNA, 22 bp, ARTIFICIAL
  PCR primer
cgccactacaaacatcggatgt

SEQ ID NO: 24 - DNA, 21 bp, ARTIFICIAL
  PCR primer
ccgcttccagaacttctttcc

SEQ ID NO: 25 - DNA, 22 bp, ARTIFICIAL
  PCR primer
tagggaactttgggtctgacag

SEQ ID NO: 26 - DNA, 25 bp, ARTIFICIAL
  PCR primer
ctgccactctaattgtcaatgtgaa

SEQ ID NO: 27 - DNA, 20 bp, ARTIFICIAL
  PCR primer
ttttccggcacagtagcaaa

SEQ ID NO: 28 - DNA, 26 bp, ARTIFICIAL
  PCR primer
gagggagaagtccctcagtgatgtga

SEQ ID NO: 29 - DNA, 26 bp, ARTIFICIAL
  PCR primer
ccttcgaggacttctagacatattcc